(19) United States
(12) Patent Application Publication
(10) Pub. No.: US 2020/0261707 A1
(43) Pub. Date: Aug. 20, 2020

(54) SYSTEM AND METHOD FOR DELIVERING THERAPEUTIC AGENTS TO THE UTERINE CAVITY

(71) Applicant: Gynion, LLC, Trumbull, CT (US)

(72) Inventors: Oleg Shikhman, Trumbull, CT (US); Steven R. Goldstein, New York, NY (US)

(73) Assignee: Gynion, LLC, Trumbull, CT (US)

(21) Appl. No.: 16/818,102

(22) Filed: Mar. 13, 2020

Related U.S. Application Data
(63) Continuation-in-part of application No. 16/523,989, filed on Jul. 26, 2019.
Application No. 16/523,989 is a continuation of application No. 15/803,415, filed on Nov. 3, 2017, now Pat. No. 10,485,962.
(60) Provisional application No. 62/421,853, filed on Nov. 14, 2016.
(60) Provisional application No. 62/824,390, filed on Mar. 27, 2019.
(60) Provisional application No. 62/843,921, filed on May 6, 2019.

(51) Int. Cl. A61M 31/00; A61B 18/06

(52) U.S. Cl. CPC A61M 31/002; A61B 18/06; A61B 2018/00559; A61M 2205/15; A61M 2210/1425

(57) ABSTRACT

An apparatus for delivering an agent to a uterine cavity of a patient for endometrial ablation including a first passage for passage of the agent into the cavity of the patient and a second passage for aspirating the agent from the uterine cavity, wherein the agent is injected at an increased pressure and is injected simultaneously with aspiration of the cavity. A cavity integrity check prior to injection of the agent can be conducted with the apparatus.

This application is a continuation in part of application Ser. No. 16/523,989, filed Jul. 26, 2019, which is a continuation of application Ser. No. 15/803,415, filed on Nov. 3, 2017, now U.S. Pat. No. 10,485,962, which claims priority from provisional application Ser. No. 62/421,853, filed Nov. 14, 2016, and this application claims priority from provisional application Ser. No. 62/843,921 filed May 6, 2019, and provisional application Ser. No. 62/824,390, filed Mar. 27, 2019. The entire contents of each of these applications are incorporated herein by reference.

### BACKGROUND OF THE INVENTION

### 1. Field of the Invention

This application relates to a system and method for delivering therapeutic agents to a patient and, more specifically, to delivering agents to a body cavity such as a uterine cavity for endometrial ablation.

### 2. Background

Heavy Menstrual Bleeding (HMB) is excessive bleeding from the vagina of over 80 mL of blood per period. Heavy periods can cause pain and discomfort and increase the risk of iron-deficiency anemia. Acute excessive bleeding can lead to hemodynamic instability, requiring hospitalization for fluid volume management, blood transfusion, and/or intravenous estrogen. This condition has a significant negative impact on woman's sexual functioning, mental well-being and overall health.

Studies have shown that Heavy Menstrual Bleeding affects approximately 1 in 3 women in their lifetime. This is over 200 million women worldwide. In the U.S. alone, there are ten million women suffering from HMB with 200,000 newly diagnosed women each year. The conservatively estimated annual direct economic cost of HMB in the US is approximately $1-1.55 billion and indirect cost is $12-36 billion.

There are four groups of treatment options that are currently available for treating HMB: 1) Dilatation and Curettage (D&C); 2) Hysterectomy; 3) Intrauterine device (IUD) and 4) Global endometrial ablation (GEA) devices. Each of these treatments has significant disadvantages. Dilation and Curettage offers a short-term relief and has a high risk of perforations. This option is not in wide use. Hysterectomy is a surgical removal of the uterus, which involves major surgery done under general anesthesia. Due to its invasive nature, high costs and risks, the number of these procedures has dropped over 50% in the last decade. Intrauterine devices, such as the Bayer HealthCare′ “Mirena” IUD, are not highly effective and have significant hormonal side effects. Yet, use of the Mirena IUD to control heavy menstrual bleeding in women seeking contraception has increased in popularity due to ease-of-use and relatively low cost of this treatment option. Global Endometrial Ablation devices, such as the Hologic “NovaSure” and the Boston Scientific “Genesys HTA”, are currently being utilized to ablate endometrium. The procedure can be done in a hospital setting or in the office. The procedure has demonstrated high efficacy, but is rather complex for in-office use and relatively expensive. Thus, GEA and IUD devices are the primary options for HMB treatment that are currently offered.

Endometrial ablation techniques, which have evolved as an alternative to hysterectomy, (e.g., laser, resecting loop with electric current, electric rollerball, thermal fluid-filled balloon, radiofrequency, freezing, heated saline) remove some of the lining of the uterus in an attempt to control excessive bleeding. After endometrial ablation, pregnancy is not likely to occur.

The early techniques of endometrial ablation, introduced in the 1980s and still used today (although much less commonly) involve the use of a hysteroscope with either a “rollerball” or wire loop through which electrical heat travels to remove (resection) the endometrial lining. After the uterus is filled with fluid to enlarge it for better viewing, the surgeon moves the rollerball back and forth across the lining or uses the wire loop to shave off the tissue. Potential risks of this ablation method include infection, perforation of the uterus, cervical laceration, and fluid overload.

In 1997, the Food and Drug Administration (FDA) approved ThermaChoice, the first non-hysteroscopic ablation device to treat excessive uterine bleeding (menorrhagia) due to benign (non-cancerous) causes. The Gynecare ThermaChoice Uterine Balloon Therapy System has a balloon that is inserted through the neck of the cervix and into the uterus. Through a catheter connected to a controller console, the balloon is inflated with fluid and heated to 188° F. (87° C.) for 8 minutes to destroy the uterine lining.

In 2001, the FDA approved three more similar devices. These devices are to be used only in women who have not yet reached menopause and whose child-bearing is completed. The BEI Medical Systems Hydro ThermAblator delivers heated saline solution into the uterus. The heated saline solution is delivered using hysteroscopic guidance. The heated solution destroys the uterine lining in about ten minutes. The CryoGen Her Option Uterine Cryoblation Therapy System uses a cryoprobe capable of producing temperatures down to minus 148° F. (minus 100° C.) at the tip. This extreme cold is applied to the tissue for ten minutes to freeze and destroy the uterine lining. Ultrasound is used to guide and monitor the procedure.

Currently available GEA treatment options are expensive and complex. As a result, only 15.8% of patients received a therapeutic procedure within twelve months, post diagnosis. Studies also show that 38% of women with HMB undergo a hysterectomy, which is a major surgery, without even being offered less invasive alternatives. These results show that physicians and patients are well-aware of these limitations and reluctant to use these treatment options.

There is a need for a non-invasive, easy-to-use (short learning curve), and effective device for treating HMB. It would further be advantageous to provide such treatment with a low cost device and low procedural costs. This would enable treatment of the patient population that currently remains untreated due to clinical and economic limitations of the current options. It would also be advantageous if such device ensured that the therapeutic agent is safely delivered to the endometrium in the uterine cavity.

### SUMMARY

The present invention overcomes the deficiencies and disadvantages of the prior art. The present invention advantageously provides in preferred embodiments an apparatus for endometrial ablation that is easy to use, economical and controls the pressure of therapeutic agent applied to the endometrium. The apparatus of the present invention also in preferred embodiments apply a pre-check of the uterine cavity to ensure it is sealed before application of the therapeutic agent, thereby preventing exposure to the agent in other areas of the body. The therapeutic agent is preferably injected to maximize the surface of exposure of the endometrium to the agent (preferably the entire surface of the endometrium will be exposed) to the agent while preventing leakage from the uterine cavity to other areas of the body.

In accordance with one aspect of the present invention, an apparatus for delivering an agent, such as a therapeutic agent, to a body cavity of a patient, such as a uterine cavity, is provided comprising a first passage for passage of the agent into the cavity of the patient, the first passage having an opening for exit of the agent, and a second passage for aspirating the agent from the cavity. The agent in these embodiments is injected at an increased pressure and is injected simultaneously with aspiration of the cavity.

In some embodiments, the second passage has a plurality of perforations to provide a plurality of entrance openings for passage of the agent into the apparatus during aspiration of the cavity. In some embodiments, the second passage aspirates gas, such as air bubbles/air pockets from the body cavity prior to injection of the agent and/or during injection of the agent.

In some embodiments, a distal portion of the second passage has a looped configuration, the loop having a first condition for delivery and a second expanded condition for placement (use) within the body cavity. In some embodiments, a tubular structure extends through a lumen of an elongated member of the apparatus and forms the looped configuration distal of the elongated member, wherein the tubular structure extends from the lumen and forms the loop terminating at an end which is distal of or alternatively aligned with a distalmost edge of the elongated member.

In some embodiments, a fluid is injected into the body cavity prior to injection of the agent to conduct a cavity integrity check to assess the presence or absence of leakage from (out of) the cavity, and the agent is injected at a pressure less than or equal to the pressure of injection of the fluid.

In some embodiments, the fluid for the cavity integrity check has a surface tension less than or equal to a surface tension of the agent and/or a viscosity less than or equal to a viscosity of the agent.

In some embodiments, the apparatus includes a line connectable to a module, the module controlling a time period of injection of the agent so the agent is injected for a preset period of time. In some embodiments, the injection module and catheter can be all-in-one, e.g., part of the catheter.

An injection module can be provided in some embodiments as a separate unit for use with the apparatus rather than integral or part of the apparatus. The injection module can include one or more of a pressure controller to control pressure, a pressure controller to control aspiration, a pressure measurement device, a flow controller to control flow and a timer to indicate/control a time period of aspiration and injection. In some embodiments, the injection module automatically transitions to aspiration of the cavity and/or injection of the agent if the absence of a leakage is assessed.

In accordance with another aspect of the present invention, an apparatus for delivering an agent, such as a therapeutic agent, to a body cavity, such as a uterine cavity, of a patient is provided comprising an elongated member having a channel for passage of the agent into the cavity of the patient, the channel having a distal opening. An expanding member extends distally of the elongated member, the expanding member having a first condition for delivery and a second expanded condition for placement (use) within the cavity. The expanding member has a plurality of perforations to provide a plurality of entrance openings for passage therein during aspiration of the cavity.

In some embodiments, the expanding member comprises a tubular structure forming a loop distal of the elongated member in the second condition.

In some embodiments, a fluid is injected into the cavity prior to injection of the agent to assess the presence or absence of leakage in the cavity, and the agent is injected at a pressure less than or equal to the pressure of injection of the fluid.

In some embodiments, aspiration of the cavity aspirates gas bubbles and/or gas pockets prior to injection of the agent and aspirates the agent after injection into the cavity.

In accordance with another aspect of the present invention, a method for injecting a therapeutic agent into a cavity of a patient, such as a uterine cavity, is provided comprising:
a) checking the integrity of the cavity to determine if there is leakage from the cavity;
b) aspirating the cavity to remove gas (e.g., bubbles); and
c) injecting the therapeutic agent into the uterine cavity under controlled pressure simultaneously with aspirating the cavity.

In some embodiments, the agent is injected into the cavity for a pre-set period of time.

In some embodiments, the method further comprises the steps of a) leaving the agent in the cavity for a preset period after injection into the cavity (a dwell period); and b) after the pre-set period evacuating the agent from the body cavity.

In some embodiments, if no leakage is assessed by checking the integrity of the cavity, the injection of the agent is automatically initiated. In other embodiments, if no leakage is determined by checking the integrity of the cavity, a user actuates a valve to open a fluid line for injection of the agent.

In accordance with another aspect of the present invention, an apparatus for delivering a therapeutic agent to the uterine cavity of the patient is provided having an elongated member having a fluid channel for passage of the agent into a uterine cavity of a patient into contact with the endometrium. The fluid channel has an opening. An expandable member extends distally of the elongated member and has a plurality of perforations to provide entrance openings for passage of the agent from the uterine cavity into the expandable member. In preferred embodiments, the therapeutic agent is a chemical agent for endometrial ablation.

In some embodiments, the injection and suction, e.g. inflow and outflow, can be through the same passage/channel/expandable member. An automated controller can control/regulate the inflow and outflow to reverse the flow.

In some embodiments, the expanding member has a tubular looped structure having a first condition for delivery having a first transverse dimension and a second condition for placement within the uterine cavity having a second transverse dimension greater than the first transverse dimension.

In accordance with another aspect of the present invention, an apparatus for delivering a therapeutic agent to the uterine cavity of the patient is provided with an elongated member having a fluid channel for passage of the therapeutic agent into the uterine cavity into contact with the endometrium. The fluid channel has a distal opening. An expandable member extends distally of the elongated member and has at least one perforation to provide for aspiration of the therapeutic agent from the uterine cavity. An infusion line passes a fluid into the uterine cavity to assess leakage to determine integrity of the uterine cavity prior to passage of the therapeutic agent into the uterine cavity. The fluid and the agent are injected at a controlled pressure. In preferred embodiments, the therapeutic agent is a chemical agent for endometrial ablation.

In some embodiments, the expandable member is a tubular looped structure having a first transverse dimension in a first condition for insertion into the uterine cavity and a second transverse dimension in a second condition for use (placement) within the uterine cavity, the second transverse dimension being greater than the first transverse dimension.

In accordance with another aspect of the present invention, a system for delivering a therapeutic agent to the uterine cavity of the patient is provided having an elongated member having a fluid channel for passage of the agent into contact with the endometrium and an expandable member extending distally of the elongated member. The expandable member has a plurality of perforations to provide entrance openings for passage of the agent from the uterine cavity during aspiration through the entrance openings. A module controls the pressure of the agent and aspiration and the time period of injection. The module can also control the pressure of fluid injected through the elongated member into the uterine cavity to assess the presence or absence of leakage in the uterine cavity. A flow control mechanism such as a valve in the module or on the handle from which the elongated member extends can be provided to open and close the lines for the pressurized agent, aspiration and pressurized fluid. A pump that turns on and off to effect aspiration could alternatively be provided. Transition from injection of the fluid to assess leakage and injection of the agent can be user controlled or alternatively automatic. Pressure gauge(s), relief valves, flow meter(s) and/or timer(s) can be provided in the module. The module can be part of the catheter or a separate unit.

In some embodiments, the expandable member is formed from a tubular structure which forms a looped configuration when expanded.

In accordance with another aspect of the present invention, a module for controlling fluid flow to a uterine cavity for an endometrial ablation procedure is provided, the module comprising a pressure controller, a pressure gauge and a timer. The module can be part of the catheter or a separate unit. The pressure controller controls the pressure of the therapeutic agent injected into the cavity. The timer ensures the agent is injected into the cavity for a pre-set period of time. In some embodiments, the module includes a control to transition from a cavity integrity check (to assess the presence of absence of leakage from the uterine cavity) to agent injection. The module can also control a pump for aspiration of the uterine cavity for aspirating gas, e.g., air bubbles or pockets, and/or agent from the cavity. The timer can in some embodiments ensure that after the agent remains in the uterine cavity for a pre-set period of time after injection, the vacuum is turned on to evacuate the agent from the cavity.

In accordance with another aspect of the present invention, a method for injecting a therapeutic agent into the uterine cavity of the patient is provided comprising the steps of a) checking the integrity of a uterine cavity to determine if there is leakage from the uterine cavity; b) if the integrity of the uterine cavity is confirmed, subsequently injecting the therapeutic agent into the uterine cavity under controlled pressure; and c) aspirating the cavity during injection of the agent. In some embodiments, the integrity of the uterine cavity is checked by injection of pressurized fluid and a) determining if the pressure remains constant after injection of the pressurized fluid is terminated and/or b) determining if flow of the pressurized fluid ceases prior to being turned off. In some embodiments, the pressurized fluid is used to inject the therapeutic agent. In some embodiments, the therapeutic agent is a chemical ablation agent.

In some embodiments, valves are controlled by the user to open the fluid, aspiration and agent lines. In other embodiments, valves are automatically actuated to open the fluid, aspiration and agent lines.

### BRIEF DESCRIPTION OF THE DRAWINGS

So that those having ordinary skill in the art to which the subject invention appertains will more readily understand how to make and use the apparatus disclosed herein, preferred embodiments thereof will be described in detail hereinbelow with reference to the drawings, wherein:

FIG. 1 is a cross-sectional view of a first embodiment of the endometrial ablation apparatus of the present invention;

FIG. 2 is a cross-sectional view of an alternate embodiment of the apparatus of the present invention;

FIG. 3 is a cross-sectional view of another alternate embodiment of the apparatus of the present invention having a pressure reduction feature;

FIG. 4 is a cross-sectional view of another alternate embodiment of the apparatus of the present invention having a balloon reinforcement feature;

FIG. 5 is a cross-sectional view of another alternate embodiment of the apparatus of the present invention having apertures in the shaft for dispensing the therapeutic agent;

FIG. 6 is a cross-sectional view of another alternate embodiment of the apparatus of the present invention having a double walled balloon to form a space for the therapeutic agent within the wall;

FIG. 7A is a cross-sectional view of the double walled balloon showing the internal wire and FIG. 7B illustrates one method to form the double balloon of FIG. 6;

FIGS. 8A and 8B are cross-sectional views of alternate embodiments showing the configuration of the wires so that the sides of the balloon are spaced to increase thickness;

FIG. 9 is lateral cross-sectional view of the uterus showing the balloon of FIG. 8A inside the uterine cavity with a thickness to ensure contact with the wall of the uterine cavity;

FIGS. 10A, 10B, 10C, and 10D are cross-sectional views of alternate embodiments of the apparatus of the present invention having a plurality of perforated tubes;

FIG. 11 is a cross-sectional view of another alternate embodiment of the apparatus of the present invention having a plurality of plugs to seal the uterine cavity;

FIG. 12 is a cross-sectional view of another alternate embodiment of the apparatus of the present invention having a balloon width indicator;

FIG. 13 is a cross-sectional view of another alternate embodiment of the apparatus of the present invention having an enlarged balloon;

FIG. 14A is a plan view illustrating the apparatus of FIG. 2 with an output suction tube and a fluid input tube, and further showing the actuator in the first position wherein the dispensing member is confined within the outer tube;

FIG. 14B is a view similar to FIG. 14A with the actuator in the second position to expose the dispensing member to allow expansion within the uterine cavity;

FIG. 15 is a plan view of an alternate system of the present invention having a syringe for injecting fluid to check the integrity of the uterine cavity and a syringe to inject an ablative agent;

FIG. 16 is a perspective view of an alternative embodiment of the present invention system showing a delivery catheter and the console (injection module) containing the selector switch for controlling fluid flow;

FIG. 17 is a schematic view of the system of FIG. 16;

FIG. 18 is a schematic view similar to FIG. 17 except showing an alternate embodiment for placement of the switch;

FIG. 19 is a perspective view of an alternate embodiment of the apparatus of the present invention showing the expandable member in the expanded condition;

FIG. 20A is a perspective view of the shaft assembly of the apparatus of FIG. 19;

FIG. 20B is a perspective view of the shaft assembly of the apparatus of FIG. 19 having an alternate cervical plug;

FIG. 21 is a cross-sectional view of the shaft assembly of FIG. 20;

FIG. 22 is enlarged view of the area of detail of FIG. 21;

FIG. 23 illustrates the distal portion of the apparatus of FIG. 19 inserted into the uterine cavity, the sheath shown in the advanced position so the expandable member is in the non-expanded position (condition);

FIG. 24 is a view similar to FIG. 23 showing the expandable member in the expanded position (condition) within the uterine cavity;

FIG. 25 is a perspective view of the pinch valve assembly of the apparatus of FIG. 19 for opening and closing the fluid lines in accordance with one embodiment of the present invention;

FIG. 26 is a close up perspective view of one of the pinch arms of FIG. 25;

FIG. 27 is a front perspective view of the cam plate of the pinch valve assembly of FIG. 25 which interacts with the pinch arms;

FIG. 28 is a perspective view showing two vials attached to the handle assembly of the apparatus;

FIG. 29 shows an alternate embodiment of a valve assembly for the apparatus of FIG. 19 having an additional valve;

FIG. 30 is a perspective view of an injection module in accordance with one embodiment of the present invention equipped with a flow meter and a pressure gauge for use with the apparatus of FIG. 19;

FIG. 31 is a perspective view of an alternate embodiment of a system of the present having two bottles separated from the handle assembly;

FIG. 32 is a perspective view of an alternate embodiment of a system of the present invention similar to FIG. 31 but having two CO2 sources;

FIG. 33 is a pneumatic diagram of an injection module that is powered by a CO2 source and uses a Venturi vacuum pump in accordance with one embodiment of the system of the present invention;

FIG. 34 is a pneumatic diagram of the system in accordance with one embodiment of the system;

FIG. 35 is a block diagram of the injection module and the apparatus;

FIG. 36 is a flow diagram showing the steps of treatment in accordance with an embodiment of the system of the present invention; and

FIG. 37 is a flow diagram showing the method of treatment in accordance with a method of the present invention.

### DETAILED DESCRIPTION OF PREFERRED EMBODIMENTS

The present invention in preferred embodiments provides a chemical global endometrium ablation apparatus for the treatment of Heavy Menstrual Bleeding (HMB). The apparatus (also referred to herein as the device, catheter, or delivery catheter) advantageously performs one or more multiple functions including: 1) expanding an expandable member inside the uterine cavity; 2) providing a cavity integrity checking feature to ensure absence of perforations, that the fallopian tubes are closed and the cervical canal of the uterine cavity is sealed prior to injection of the chemical agent and/or 3) injecting at sufficient pressure the chemical agent at a desired controlled pressure for application of the agent to the endometrium (the tissue layer that lines the inside of the uterus wall). The agent in some embodiments is injected through the expandable member; in other embodiments it is injected through the catheter out of a distal opening. Further, in some embodiments, the agent is injected during application of suction to the body cavity. The various embodiments are discussed in detail below.

The therapeutic agent is preferably injected at a pressure to maximize the surface of exposure of the endometrium to the agent (preferably the entire surface will be exposed) while preventing leakage to other areas. In the absence of perforations, and when the cervical canal is sealed by the device, the uterine cavity should be sealed as long as injection pressure will remain below the pressure that is necessary to push fluids into fallopian tubes. Therefore, in these embodiments there are two pressure limits: 1) the upper limit to prevent leakage and 2) the lower limit to assure complete exposure.

Further, to ensure that the therapeutic agent does not leak into the fallopian tubes, a cavity integrity check is conducted in some embodiments to inject a fluid at a given pressure. If the integrity check detects no leakage, then the therapeutic agent is injected at an equal or lower pressure. Moreover, the fluid for the integrity check can be selected so its properties enable easier passage into the fallopian tubes than the therapeutic agent. This is discussed in more detail below.

The apparatus can also include one or more sealing members to seal the cavity from leaks of the chemical agent or air through the cervix and/or into the fallopian tubes.

In some embodiments of the present invention, the apparatus includes a control which is operable by the clinician to achieve the foregoing functions in the single device, as discussed in more detail below.

The present invention in alternative embodiments also includes systems that instead of a manifold include separate devices, e.g., syringes, connectable to the apparatus downstream of the handle to inject fluid and gas through the fluid channel in the apparatus for agent delivery and/or cavity integrity checking. These embodiments are also discussed in detail below.

The systems of the present invention can also include a console or injection module that controls pressure and/or measures pressure flow and other parameters, discussed in detail below.

The apparatus is designed in preferred embodiments to deliver the therapeutic agent in the form of a liquid chemical agent (substance) for a chemical endometrial ablation procedure. One cauterizing agent which can be used is an acid such as trichloroacetic acid (TCA). Derivatives of trichloroacetic such as bichloroacetic acid, and other substances such as silver nitrate, and derivatives of silver nitrate can also be utilized in certain embodiments. TCA is a chemical agent that denatures on contact with protein and causes chemical cauterization on contact with tissue, but does not spread beyond where it is directly applied. Additionally, instead of chemical agents, other therapeutic agents can be delivered, the devices/systems herein not being limited to chemical endometrial ablation as for example a specially formulated substance, such as a therapeutic agent in the form of a drug with a pharmaceutical formula that is specially formulated for this application can be utilized. The therapeutic agent could also be in the form of a gas, gel, powder or granules that are mixed or dissolved in the cavity. Additionally, the apparatus can be used to inject a diagnostic fluid, for example saline or sterile water, in procedures such as genomic lavage.

Additionally, although disclosed for use within the uterine cavity for endometrial ablation, the apparatus and systems disclosed herein are not so limited and can be used for treatment of other conditions and/or for treatment in other body areas or body spaces (cavities lumnes) of the patient.

As used herein, the term ‘proximal” denotes the portion of the device closer to the user and the term “distal” denotes the portion of the device further from the user. Also, the terms apparatus and device are used herein interchangeably.

As used herein, the term “about” and the term “substantially” means±(plus and minus) 20% of the stated numeric value.

As used herein, as is convention, the term “fluid” includes a liquid or gas.

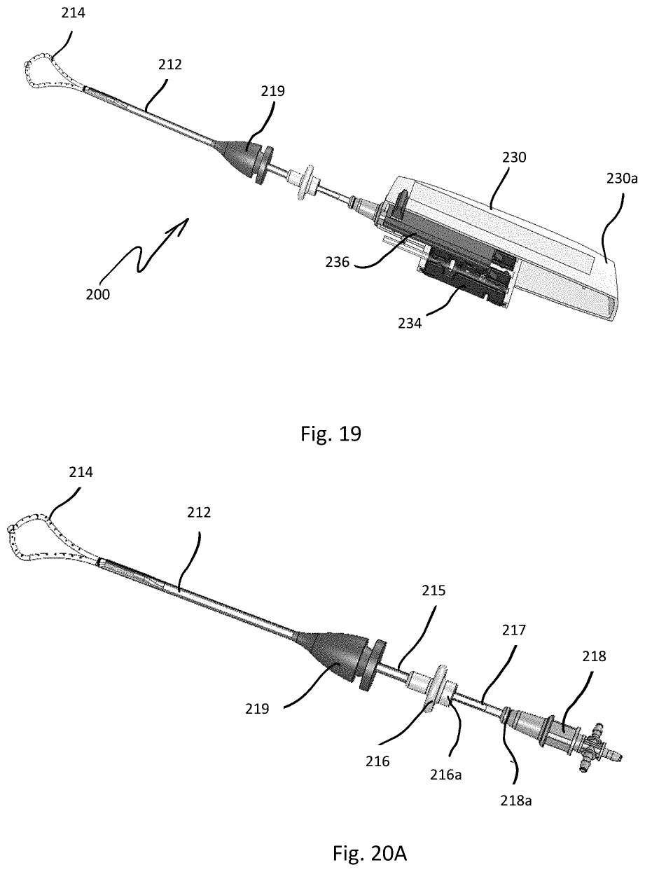

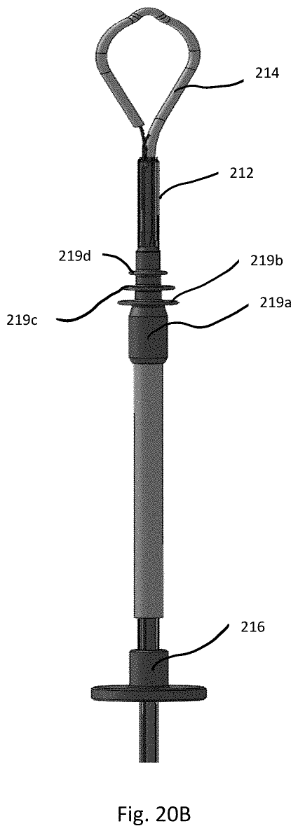

Turning now to the expandable (expanding) member (element/component) of the apparatus of the present invention, various embodiments are shown in FIGS. 1-13. The expandable member in FIGS. 1-13 forms a dispensing or dispersing member for enabling passage of the injected agent into the uterine cavity into contact with the endometrium. In the alternate embodiments of FIGS. 19-24, the expanding member, rather than dispensing the therapeutic agent, is used for evacuation, e.g., suction of the body cavity. The agent is injected through the catheter and exits the distal opening in the catheter, independent of the expanding member.

It should be appreciated that the “dispensing members” in FIGS. 1-13 can alternatively be used for aspiration/evacuation and the expanding member in FIGS. 19-24 can alternatively be used for agent injection. Note it is also contemplated that in some embodiments, both the dispensing/expanding member and the distal opening in the catheter can be used for one or both of evacuation (aspiration) and agent injection. Inflow (for agent and/or fluid for the cavity integrity check) and outflow (for aspiration) can be through different or the same channels (passages) in the catheter.

The embodiments of FIGS. 1-13 will first be discussed for dispersing the agent through the dispensing member. As noted above, alternatively the agent could be injected through a distal opening in the catheter and the identified “dispensing member” functions instead as an “evacuation/aspiration member.” Thus, it can be denoted as an “expanding member” which can include one or both of an agent dispensing function and a cavity evacuation function.

The dispensing member in these embodiments of FIGS. 1-13 is expandable and configured to maximize the area of endometrial tissue that is exposed to the agent injected through the perforations in the member. In some embodiments, the dispensing element expands to a size so it is adjacent but not in contact with the endometrium to provide space for flow of the agent from the dispensing member to the endometrium. In alternate embodiments, the dispensing member can be configured to expand to the contour of the uterus and into contact with the endometrium to fully fill the uterine cavity. In other embodiments, the dispensing member is not expandable. Note FIGS. 1-13 show the distal end of the apparatus which has the expandable dispensing member it being understood that the proximal end, e.g., handle is omitted from these drawings. A handle such as handle 230 of FIG. 19 or the handle of FIG. 16 could be utilized for example. The expandable dispensing member can be in the form of a perforated (pierced) balloon, a pad made out of porous material, a porous material such as foam or a sponge-like material, a foam inside a balloon, or in the form of one or more apertured tubes. The therapeutic agent can then flow though the perforations or pores in the expanded dispensing member and into contact with the endometrium. For delivery, the dispensing member is in a collapsed or low profile (smaller transverse dimension) condition (position) and expands to a higher profile (larger transverse dimension) condition (position) within the uterine cavity.

When an agent such as TCA utilized, it is in preferred embodiments is intended to completely ablate the endometrium and also not only to contact the endometrium but to penetrate deeper into the myometrium. The application of TCA as disclosed herein can be controlled, e.g., timed, as disclosed herein, to assure the TCA doesn't penetrate myometrium to deep so as to come too close to serosa.

Referring now in detail to the drawings wherein like reference numerals identify similar or like components throughout the several views, a first embodiment of the expandable member is shown in FIG. 1. The apparatus 10 includes a balloon 11 having a plurality of perforations 12 extending through the wall to enable communication of fluid from the interior of the balloon to the uterus B, i.e., endometrium A. The perforations 12 are shown dispersed throughout the wall of the balloon to provide sufficient application of therapeutic agent, e.g., chemical agent, to the endometrium. The perforations can in some embodiments be uniformly positioned to provide uniform application of agent so the agent is spread evenly through the endometrial cavity for uniform adequate ablation. In some embodiments, a pre-calculated volume of agent at a controlled pressure is injected into the balloon. In other embodiments, the agent is injected without volume calculation until a target controlled pressure within the uterine cavity is achieved.

Note that as an alternative to the balloon 11, as well as an alternative to the other embodiments of balloons disclosed herein, a foam material, sponge, or other material that expands and has perforations or pores to enable application of the agent to the lining of the uterus (endometrium) can be utilized. Additionally, the balloon can be filled with a foam. In any of these embodiments, the size of the holes of the balloon (or foam material, sponge, etc.) can be varied to control the flow and volume of the agent in different areas of the balloon.

Balloon 11 may also include a support wire 14 which expands the balloon 11, i.e., forces the balloon 11 open. The wire 14 facilitates expansion to maximize the exposure area of the balloon with respect to the endometrium. The wire 14 can be made of material with sufficient springiness or of shape memory material so that when deployed from the outer tube or sheath 19, it moves from its collapsed or compressed condition inside sheath 19 to an expanded position of larger transverse dimension shown in FIG. 1. Note the wire 14 is shown positioned along the entire periphery of the balloon to expand the balloon 11.

The balloon 11 and supporting/expanding wire 14 are supported, e.g., attached, at a distal end on shaft 18 which is movable relative to sheath 19. That is, for delivery to the uterus, the wire 14 and balloon 11 are retained inside the sheath 19 as the shaft 18 is retracted within the sheath 19. To deploy the balloon 11 and wire 14, the sheath 19 is retracted, the shaft 18 is advanced distally or both the sheath 19 and shaft 18 are moved relative to one another so that the balloon 11 and wire 14 are distal of the sheath 19 and exposed from the confines of the sheath wall, the term “relative movement” or “movement relative to” encompassing these three alternatives. Exposure of the balloon 11 and wire 14 from the confines of sheath 19 enables expansion of the balloon 11 due to expansion of the wire 14. The agent is injected through channel or lumen 18a in shaft 18, the channel 18a having a distal opening in communication with the interior of balloon 11 so the agent (e.g., chemical ablative substance/agent) flows through the channel, exiting the distal opening into the interior of balloon 11. In addition to the channel for delivery of the agent to the dispensing member, additional channels could be provided for use for other purposes, such as a separate channel for inflation of the balloon, insertion of other instruments, tools, scope, camera, etc.

Markings can be provided on sheath 19 to indicate the depth of insertion of the apparatus 10 into the uterine cavity. Markings can also be provided on shaft 18 to indicate the extent of exposure from the sheath 19. Such marking on the sheath and/or shaft can also be provided in the other embodiments disclosed herein. The outer sheath 19 is configured for ease of insertion through the cervix and in some embodiments is sized such that it would require no or minimum dilation of the cervix prior to insertion.

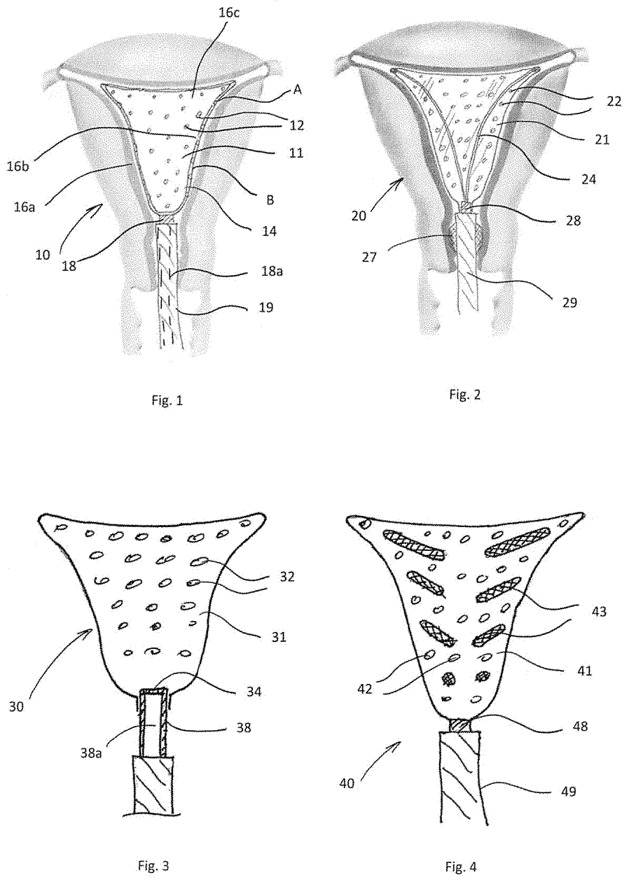

The structural wire 14 as shown in FIG. 1 extends along the periphery of the balloon 11, with portions 16a, 16b extending distally from the distal end of shaft 18 and a portion 16c extending transversely to portions 16a, 16b. A single wire shaped as shown or separate wires attached in the illustrated configuration can be utilized. In alternate embodiments, the wire 14 can have other configurations/shapes and/or be positioned in other regions of the balloon such as shown for example in the embodiment of FIG. 2. In this embodiment of FIG. 2, wire 24 of device 20 is positioned more inwardly of the periphery of balloon 21. The balloon 21 of FIG. 2 is otherwise identical to balloon 11 of FIG. 1, e.g., has perforations 22, is supported on shaft 28 which is slidable with respect to sheath 29, etc., so for brevity further description is not provided as the function and structure of the elements of FIG. 1, e.g., shaft, sheath, balloon, etc., are fully applicable to the embodiment of FIG. 2. Note that the wire is shaped to facilitate full deployment of the balloon (dispensing member) 21. That is, wires 14 and 24 are utilized to assure that the dispensing member is expanded to maximize the exposure area as the wire reinforces the balloon and is used to force the balloon to open up. Since the uterus is a 2D organ with walls touching each other unless a force is applied to separate them and distend the cavity, the force of the wire allows the balloon to open separating the walls. The size of the holes in the pierced balloon could vary to control the flow and the volume of the substance in different areas of the balloon. A sealing member such as a balloon or plug 27 is provided around the sheath (FIG. 2) to seal the cervical canal to prevent outflow of fluid, e.g., CO2 (carbon dioxide), sterile water or saline and the therapeutic agent.

In some embodiments utilizing a structural wire to expand the balloon, the balloon expansion is independent of the therapeutic agent. In this manner, the agent dosage can be determined solely by the clinical need to effectively perform ablation or other treatment rather than requiring sufficient injection to first inflate the balloon, followed by passage through the balloon. In other words, in such embodiments, the agent is not used for balloon inflation but only for dispensing through the balloon, independent of the expansion by the internal wire. By relying on mechanical expansion, it also enables agent pressure to be minimized so excessive pressure is not applied. The balloons can be made for example of a non-compliant elastomeric material such as PET (polyethylene terephthalate), although other materials are also contemplated.

It is also contemplated that in alternate embodiments, instead of a wire to expand the balloon in the various embodiments disclosed herein, the balloon can be expanded by the pressure of the injection fluid.

In the alternate embodiment of FIG. 3, the balloon 31 (dispersing/dispensing member) of apparatus 30 is similar to balloon 11 in that it has a plurality of perforations 32 for dispensing the chemical agent. The apparatus 30 differs from device 10 in that the shaft 38 movable relative to shaft 30 has a “flow reduction” feature designated by reference numeral 34. The flow rate reduction feature 33 is shown in the form of an aperture 34 at the end of the channel (lumen) 38a within shaft 38 having a diameter less than the diameter of the shaft 38. This provides injection of the agent at a lower flow rate.

The balloons disclosed herein can include welded areas, such as areas 43 of balloon (dispensing member) 41 of device 40 shown in FIG. 4. These strips reduce balloon's distension to minimize amount of the agent needed to inflate it. That is, they keep the balloon expanded in essentially two dimensions, e.g., substantially flat. Balloon 41 has perforations 42 and is supported on shaft 48, movable relative to the sheath 49, in the same manner as shaft 18 described above. Balloon 41 can include the internal expandable wire structures, e.g., wires 14 or 24 disclosed herein.

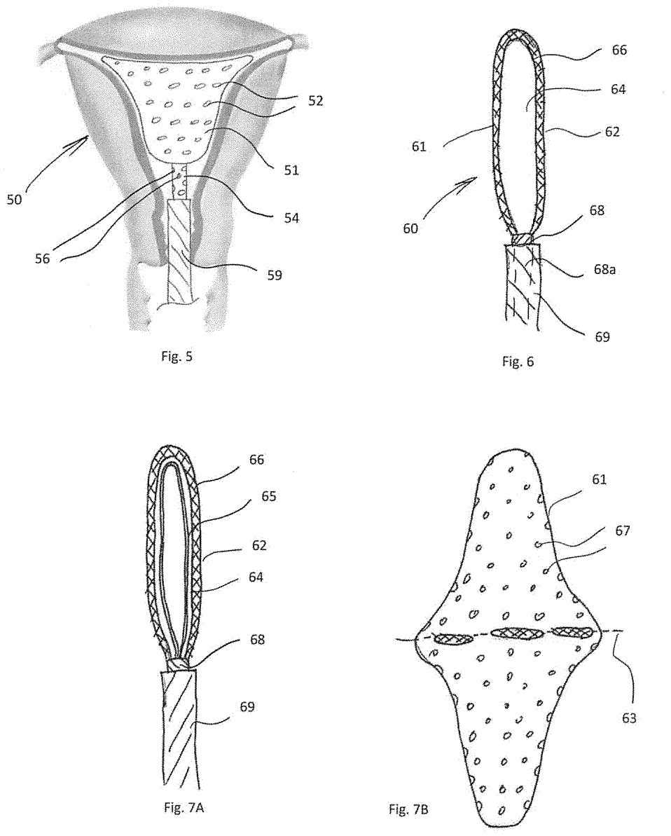

In the embodiments disclosed herein, the shaft (elongated member) could have additional perforations to maximize exposure in cases where the length of the cavity exceeds the length of the dispensing member. This is shown for example in FIG. 5 wherein apparatus 50 has a dispensing member 51 in the form of a balloon with perforations 52 like perforations 22 described above. An internal reinforcing expanding wire structure, like wire 14 or 24 described herein, can be provided within the balloon 51. Shaft 54 has a channel or lumen extending therein with an opening in communication with the interior of balloon 51 for passage of injected agent into the interior of balloon 51 for exiting perforations 52. Shaft 54 further has a plurality of perforations 56 in its side wall, proximal of the balloon 52, to provide additional flow of agent into the uterine cavity and endometrium through these side perforations. It would also potentially allow exposure of tissue within the cervical canal, which in some cases could be clinically beneficial. The shaft 54 is relatively slidable with respect to sheath (outer tube) 59 in the same manner as aforedescribed shaft 18 and sheath 19. These one or more perforations (openings) can be provided in the side wall of the shaft of the other embodiments disclosed herein. Further, the agent can exit through a distal opening in the shaft either in addition or in lieu of the side openings. The side openings can supplement agent outflow through the balloon or alternatively be in lieu of outflow through the balloon.

FIGS. 6, 7A and 7B show an alternate embodiment of a dispensing member in the form of a double walled balloon (or a balloon within a balloon) wherein the therapeutic agent is injected into the space between the wall of the balloon or the wall between the outer and inner balloon. More specifically, balloon 61 of apparatus 60 has an outer wall 62 and an inner wall 64, spaced apart sufficiently to create sufficient space/volume for the agent. The channel 68a of shaft 68 is in fluid communication with the space (balloon channel) 66 between balloon walls 62 and 64 so the agent can flow through the channel, into the space 66 and out through perforations 67 in outer wall 62. The inner space of the balloon can be filled with a fluid, i.e., a gas, e.g., air, or a liquid and/or an expanding internal wire 65 (see FIG. 7A) similar to wires 14 or 24 to aid expansion. (Fluid as defined herein including a gas or liquid). By injecting the agent only into the reduced space/volume provided between the inner and outer wall, rather than though the internal space/volume of the balloon, the injectable volume of the agent is minimized.

One possible way to form the balloon 61 is shown in FIGS. 7A and 7B wherein the balloon 61 has a fold line 63. The balloon is initially in a more flattened condition (FIG. 7B) and then folded along fold line 63 and then sealed along its periphery to join the walls of the balloon 61 to create the inner and outer spaced apart walls. The shaft 68 and sheath (outer tube) 69 are relatively slidable in the same manner as aforedescribed shaft 18 and sheath 19.

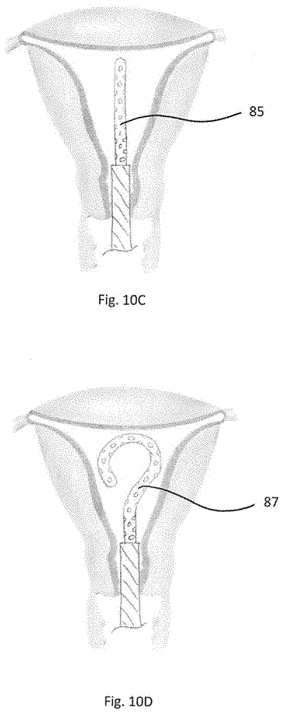

As mentioned above, as an alternative to a balloon, sponge, foam or other porous or perforated material, the dispersing/dispensing member can include one or more perforated tubes. In FIG. 10A, apparatus 70 has an elongated member (shaft) 78 coaxially positioned within outer tube or sheath 79, and slidable relative to sheath 79. Extending from shaft 78 are a network (series) of tubes including two distally directed curved side tubes 74, 75, a longitudinally extending tube 76 and a transverse tube 77, forming a closed shaped configuration as shown. The tubes 74, 75, 76 and 77 each have perforations 74a, 75a, 76a and 77a, respectively, through which the therapeutic agent exits. The agent flows through a channel in the shaft 78 and into the channel or lumen within the tubes 74-77, exiting through the perforations 74a-77a into the uterine cavity. The tubes 74 and 75 are expandable for positioning adjacent the endometrium, with tubes 76 and 77 providing additional support and/or additional openings for flow of the therapeutic agent into the endometrium. The tubes can be made of a self-expanding material or a flexible material that automatically moves to the expanded position when exposed from the sheath. Alternatively, the tubes can have an internal wire positioned therein, similar to the internal wires disclosed herein, such as wires 14 or 24, which expand when exposed from the sheath to thereby expand the tubes to the position of FIG. 10A. The network of tubes shown is only one possible arrangement, and a greater or fewer number of tubes can be provided as well as a different arrangement of tubes other than that shown as long as the therapeutic agent is sufficiently dispersed throughout the uterine cavity. The tubes could be interconnected with each other, or alternatively, they could remain not connected to each other with each tube having an independent flow of the agent from the shaft. One example of an alternate arrangement of the tubes is shown in FIG. 10B wherein only two tubes 82, 84 are provided as sufficient to fill the cavity and dispense/disperse sufficient agent. In all other respects, device 80 is identical to device 70, e.g., has tube perforations 82a 84a, shaft 88, sheath 89, etc. so for brevity discussion of these components/features is not provided. FIG. 10C illustrates an alternate embodiment with a single straight (linear) perforated tube 85 and is an example of a non-expanding dispensing member. FIG. 10D illustrates another alternate embodiment with a hook shaped perforated tube 87. Note these perforated tube configurations of FIGS. 10A-10D can also be utilized for suction in the manner described below in conjunction with the apparatus of FIGS. 19-34. With such use, suction would be applied to the cavity via the holes in the tube(s) and the agent would be injected into the cavity through the distal opening in the shaft. Thus, the discussion of the function of the apparatus of FIG. 19, along with the systems disclosed for use with the apparatus including, e.g., the module, is applicable to the tubular structures of FIGS. 10A, 10B, 10C and 10D in an alternate operation of these apparatus as the expandable tubular structures would operate as evacuation or suction members rather than dispensing members.

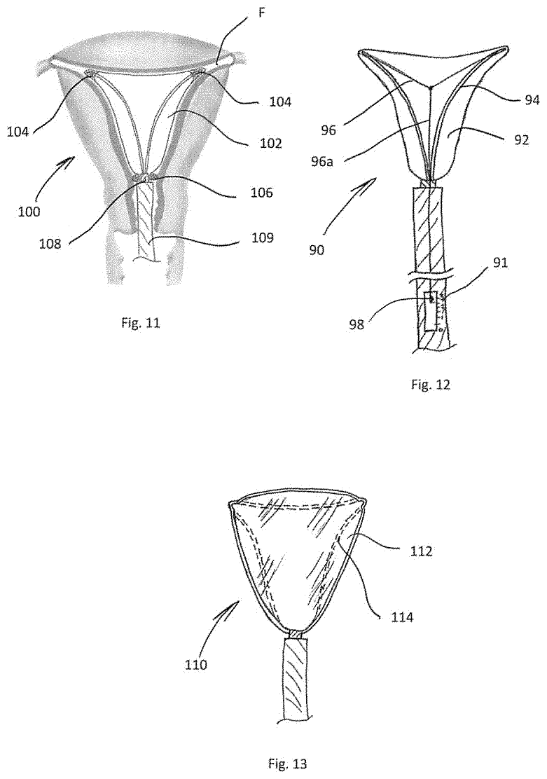

The foregoing apparatus can include in some embodiments a feature that allows users to confirm that the dispensing member has opened and see how wide it has opened. This is shown in FIG. 12 wherein width indicator of apparatus 90 has markings 91 which indicate the width of the opening of the dispensing member which is in the form of balloon 92 to ensure the balloon has fully deployed within the uterine cavity. As the internal wire 94 which expands the balloon 92 is expanded as shown, it applies a force on the indicator wire 96, pulling portion 96a distally which pulls the marker 98 distally to indicate the extent of expansion of the balloon 92 by its position with respect to numeric makings 91. Other markings are also contemplated. This feature could be useful to ensure the dispensing is fully deployed as well as helpful in determining a needed volume of the chemical agent.

Any of the devices disclosed herein can include protective plugs to prevent or minimize the flow of the therapeutic agent into the fallopian tubes/and or into the cervix. An example of such plugs is shown in FIG. 11 wherein apparatus 100 has a plug 104 at distal end regions, e.g., distal corners, of the balloon 102 to block the therapeutic (treatment) agent, e.g., the chemical agent such as TCA, from flowing into the fallopian tubes F where it could damage the fallopian tubes. A plug 106 is positioned at a proximal region of the balloon 102, attached to the outer wall of the inner shaft 108 as shown, or alternatively, attached to a proximal portion of the balloon 102, to block the flow of the agent proximally into the cervix. Alternatively, a small balloon could be utilized in place of one or more of the plugs 104, 106. The proximal plug in alternate embodiments can include an annular balloon around the sheath 109 to seal the cavity from leaks of the agent or air through the cervix. In some embodiments, the plug can be slidable along the sheath or shaft or mounted to the sheath as in FIG. 2 or FIG. 19 for example. If the cervix is sealed with such balloon or plug, and the fallopian tubes are closed or blocked and there is no uterus perforation, the entire cavity is sealed.

In some embodiments, the perforated dispensing member is configured and dimensioned so that when expanded its outer wall is close to but not necessarily in contact with the endometrium. However, it is also contemplated that the dispensing member can be configured and dimensioned so that when expanded it conforms to the contour of the uterus, thereby expanding to be in contact (abutment) with the endometrium. An example of such oversized perforated dispensing member for passage of the therapeutic agent is shown in FIG. 13 wherein the dispensing (dispersing) member 112 of apparatus 110 is in the form of a balloon. The dimensions of the balloon 112 are greater than that of the aforedescribed embodiments so as to more fully fill the uterine cavity. That is, when deployed, the dispensing member 112 will be compressed by the wall of the uterus and forced to comply/conform with the shape of the cavity. The balloon 112 could be reinforced with a structural wire 114 that is biased outward as in the wires discussed above. Such oversized dispensing member can be utilized with any of the embodiments disclosed herein. Note also the embodiments having perforated tubes could utilize tubes which expand further to come into contact (abutment) with the endometrium.

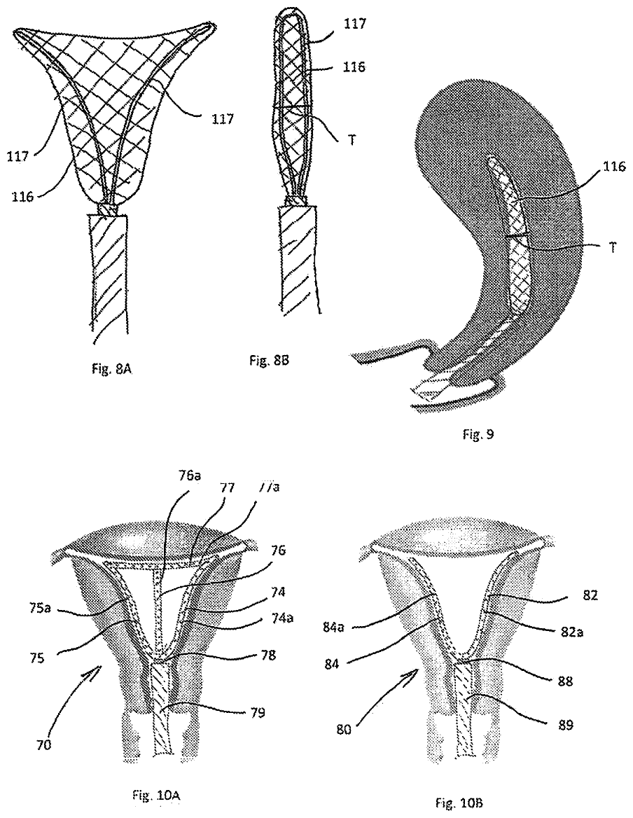

FIGS. 8A, 8B and 9 show an alternate embodiment wherein the balloon is expanded in three dimensions. In the other embodiments disclosed herein, the balloons are essentially expanded in two dimensions as they are relatively flat. In this embodiment, dispensing member (balloon) 116 has looped shaped wires 117 to expand the balloon in three dimensions, providing an increased thickness T of the balloon 116 as shown in the lateral cross-section of the uterus with the balloon inside of the uterine cavity (FIG. 9). The structural wire 117 is configured such that the sides of the balloon 116 are spaced to assure the needed thickness. The thickness of the balloon 116 is such that it facilitates contact with the wall of the uterine cavity so the wall gets exposed to the therapeutic agent even if it is injected at a low pressure.

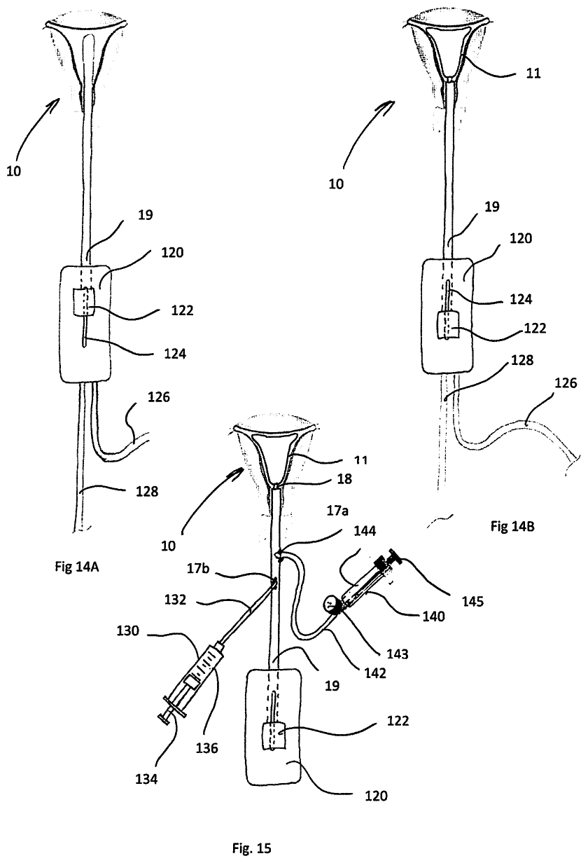

FIGS. 14A and 14B illustrate one embodiment of the actuator of the apparatus for exposing the expandable member, e.g., the dispensing/dispersing member, for expansion. This is shown utilizing apparatus 10 of FIG. 1 by way of example, it being understood that the other apparatus disclosed herein, including the various aforedescribed balloons, wire structures, perforated tubes, etc., can use the same handle and actuator of FIGS. 14A and 14B to effect exposure of the expanding member at the distal end of the shaft. Apparatus 10 has a handle 120 with a slidable actuator 122, e.g., a slidable button, movable within slot 124 of handle 120. The actuator 122 is operatively connected to the outer tube (sheath) 19 and is movable between a distal position wherein the sheath 19 is in the distal (extended) position and the balloon 11 and wire 14 are in the collapsed (reduced profile) position within the sheath 19 and a proximal (retracted) position wherein the sheath 19 is retracted to expose the balloon 11 and wire 14 for expansion to the expanded position within the uterine cavity. Alternatively, the sheath 19 could be stationary and attached to the handle, while the shaft 18 is operatively connected to the slidable actuator so that the shaft would be advanced by pushing the sliding button to the forward (distal) position to expose and expand the dispensing member. Other actuators are also contemplated such as other forms of linear actuators or rotatable actuators.

The apparatus of FIGS. 14A and 14B also include a suction line 126 and a fluid input line 128 which includes a cavity integrity checking feature. This is discussed below in conjunction with FIGS. 16-18. The agent input line can be a separate line in communication with the expandable member. Within the shaft, the line can communicate with independent channels or shared channels, e.g., the therapeutic agent input and fluid input for integrity check can be through the same channel.

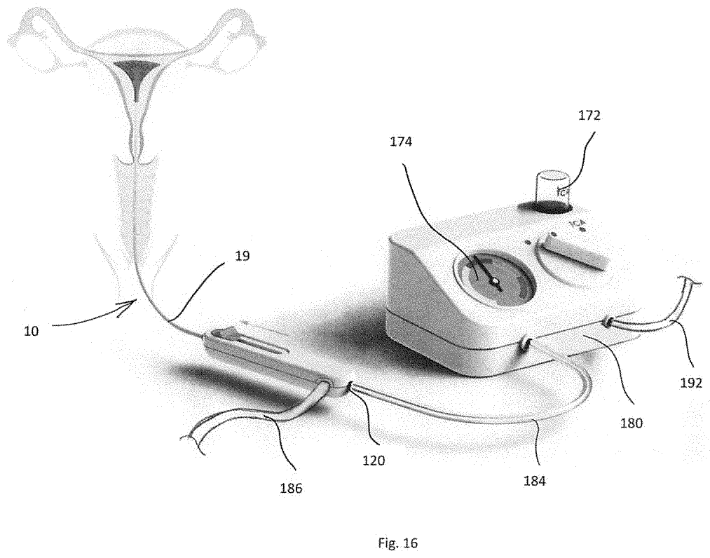

Turning now to the various systems of the present invention which include the fluid and suction lines, FIG. 15 illustrates an embodiment with a separate syringe for the uterine cavity integrity check and for the therapeutic agent. Both syringes are connected to the shaft of the apparatus downstream of the handle. FIG. 16 illustrates an alternate system wherein a separate console (module) is provided upstream of the handle with a switching mechanism for injection of the fluid for the cavity integrity check and for injection of the agent. The terms downstream and upstream as used herein refer to the direction of fluid flow into the cavity—fluid is injected and flows in a downstream direction. These systems of FIGS. 15 and 16 can be used with the various embodiments of the apparatus disclosed herein.

Turning first to the system of FIG. 15, the system includes a syringe 130 containing the therapeutic agent and a syringe 140 for checking the pressure within the uterine cavity and is shown in conjunction with the apparatus 10 by way of example, although the syringes 130, 140 can be used in the same manner in conjunction with the other apparatus disclosed herein. More specifically, syringe 140 has a tube 142 connecting the barrel 144 to the channel, e.g. channel 18a of shaft 18, which forms the fluid line. The tube 142 extends into side port 17a of the apparatus 10 for fluid communication with the channel. A pressure gauge 143 is also provided on the syringe 140 for measuring pressure within the uterine cavity. When the plunger 145 is advanced, fluid such as a gas, e.g., CO2, air, etc. or a liquid such as sterile water or saline, is advanced from barrel 144 into the line (tube) 142, into the channel of shaft 18 and out through the distal opening of the channel 18a into the uterine cavity. Intrauterine pressure is monitored as the fluid (gas or liquid) is injected. It typically takes a pressure of 60-80 mm Hg to push fluids into fallopian tubes. Since the intent is to avoid flow of the agent through the fallopian tubes, especially if a chemical ablative agent is used, in this embodiment, the cavity integrity is checked by inflating it to a pressure level that is lower than 60-80 mm Hg, for example lower than 50 mmHg. This pressure level is sufficient to create a leak of fluid if the uterine cavity is perforated. Thus, after the pressure level is achieved e.g., a preset level, the flow is ceased and the pressure of the cavity is observed utilizing pressure gauge 143. If the pressure remains constant after termination of liquid or gas input, this will signify there is no leakage, thereby indicating (with the cervical plug sealing the cavity) that there are no perforations in the uterus and that the fallopian tubes are closed and that the uterine cavity is sealed for application of the therapeutic agent. If on the other hand the pressure drops after flow ceases, this will indicate that gas or liquid is escaping and the fallopian tubes are open and/or there are perforations in the uterus, thus informing the clinician that the uterine cavity is not sufficiently sealed and the therapeutic agent should not be applied because it could leak into undesired areas. Note the syringe can be used to initially apply suction to the uterine cavity via retraction of the plunger 145 of syringe 140 to remove air pockets/bubbles prior to injection of the gas or liquid for checking the integrity of the uterine cavity. Additionally, after the cavity integrity check/test is completed, the gas or liquid can be suctioned/evacuated by reverse movement of the plunger 145 of the syringe 140. Alternatively, another syringe or suction source can be utilized. Note the above pressure levels are based on current testing, it being understood that other pressure levels are also within the scope of the invention.

The syringe 130 can be similar to syringe 140 and could be equipped with a pressure gauge and an injection line 132 which is in fluid communication with the channel 18a of shaft 18 via attachment to side port 17b of outer tube 19. Movement of the plunger 134 forces the therapeutic agent, e.g. chemical ablative agent, out of barrel 136 and into injection line (tube or shaft) 132 for passage into channel 18a and into the balloon 11, where it exits through the balloon perforations 12 into the uterine cavity to ablate the endometrium. The syringe 130 is actuated after the syringe 140 confirms the uterine cavity is sealed to ensure that the chemical ablation substance or other agent being injected does not exit the uterine cavity and damage the fallopian tubes or other areas of the body. If the syringe 130 is equipped with the pressure gauge, the injection pressure is maintained at the level equal or below the pressure level at which the integrity of the uterine cavity was tested.

The slidable actuator 122 is operatively connected to outer tube (sheath) 19 so that movement of the actuator 122 retracts outer tube 19 so the balloon 11 and internal wire 14 attached to shaft 18 are exposed from outer sheath 19 so the balloon 11, via the radial force of the wire 14, expands to the expanded position shown in the same manner as described with respect to FIGS. 14A and 14B. Note other actuators can be utilized such as rotatable actuators.

In use, the balloon (dispensing/dispersing) member 11 is expanded by proximal movement of the sheath 19 via actuator 122. After expansion of the balloon 11 and prior to injection of the chemical ablative agent (or other therapeutic agent), the syringe 140 is operated to inject gas (or liquid) though line 142 and through the channel 18a and out the distal opening of channel 18a into the uterine cavity to conduct the cavity integrity check. If the integrity of the uterine cavity is confirmed, i.e., there is no leakage into the fallopian tubes or other parts of the body from the uterus, the injected gas (or liquid) is evacuated by the syringe 140, and then the syringe 130 is actuated to advance the agent though line 132 and through the perforations 12 in expanded balloon 11 to contact, e.g., chemically ablate the endometrium (or in alternate embodiments like the FIG. 19 apparatus injected out the distal opening of channel 217b). As noted above, the other apparatus described above can be utilized with the system of FIG. 15 in the same manner as apparatus 10, i.e., dispensing member deployed by an actuator 122, cavity integrity checked by syringe 140, agent injected by syringe 130, etc. Thus, the other apparatus disclosed herein can include side ports for receipt of the syringes 130, 140 distal of the handle 120.

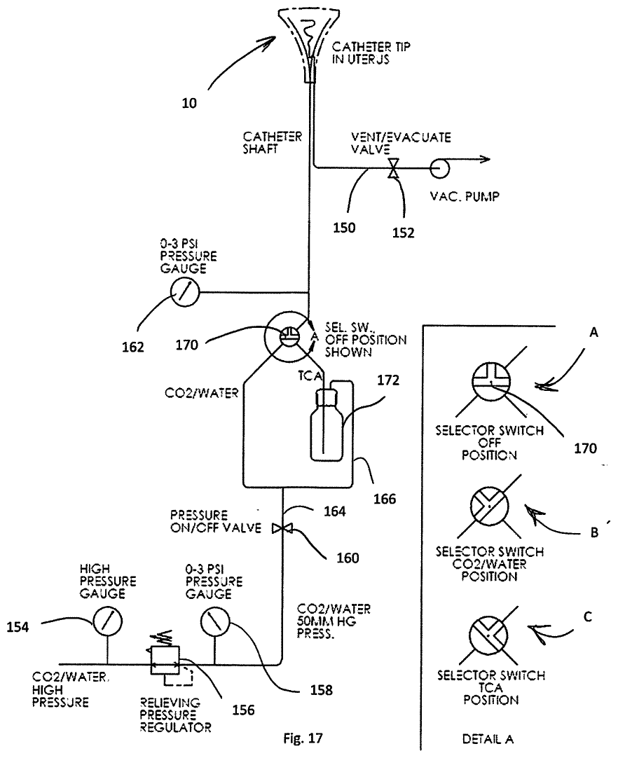

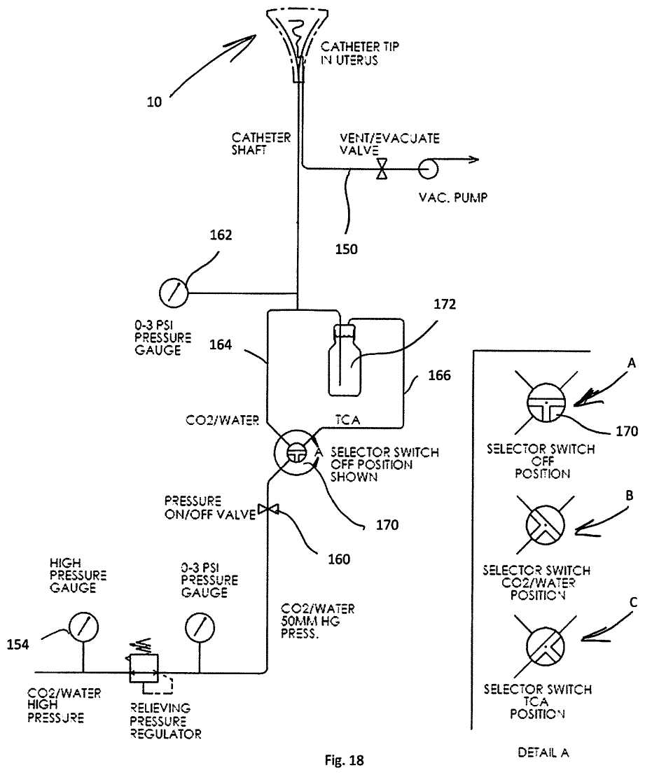

FIGS. 16-18 illustrate alternative embodiments of the system of the present invention wherein suction and injection lines extend through the handle of the apparatus. These provide an alternative approach to a cavity integrity checking feature, utilizing a single pressurized fluid source combined with the manifold to conduct the cavity integrity check and injection of the therapeutic agent. The system of FIGS. 16-18 can be used with the various apparatus (catheters) disclosed herein. The manifold is in the form of a control which is in the form of a switch which allows the user to connect the fluid injection line to multiple lines inside of the delivery system. The lines are controlled by the manifold described in conjunction with the schematic diagrams of FIGS. 17 and 18. FIG. 18 illustrates a system wherein the manifold is reusable because it is not contaminated by the therapeutic agent, e.g., chemical agent; FIG. 17 illustrates an alternative system wherein the therapeutic agent, e.g., chemical agent, flows through the manifold and therefore is disposable (not reusable) since it is contaminated by the agent. Otherwise, these systems are the same. FIG. 16 is a perspective view of the system showing the console (module) 180 which contains the selector switch 170 and supports the therapeutic agent within container 172, e.g. a jar or vial, which is connected via an injection line to the delivery channel for delivery into the uterine cavity. The console also includes a pressure gauge 174. The input line (input tube) 182 to the console 180 is for the high pressure gas or liquid into the manifold and the output line (outlet tube) 184 is for the pressurized gas or liquid or pressured agent from the manifold. Line (tube) 186 is for suction. The apparatus, e.g., apparatus 10, or other apparatus disclosed herein, is connectable to the console 180 via tube 184. If sterile saline is used for the integrity check, the console can have a receptacle to receive a container or jar of sterile saline. Note that the various apparatus and consoles can be packaged together or packaged separately.

Turning first to the embodiment of FIG. 17, the catheter shaft and expanded expandable member are shown schematically and can include any of the shafts and expandable members discussed herein. A suction line (tube) 150 (see also tube 186 of FIG. 16) communicates with the internal channel of the shaft and extends from an external vacuum pump. A vent/valve 152 turns the suction on and off. When the pump valve is in the open position, suction is applied to the uterine cavity for evacuation of the uterine cavity. The suction line can be turned on at various stages of the procedure including one or more of the following: 1) initially before the pressurized fluid is injected for performing the cavity integrity check to remove gas or air bubble pockets; 2) after the cavity integrity check to remove the liquid or gas from the uterine cavity and the catheter lines that was used for the cavity check; 3) during application of the therapeutic agent to the endometrium; and/or 4) after application of the therapeutic agent to the endometrium to remove agent from the uterine cavity.

With continued reference to the diagram of FIG. 17, a gas such as carbon dioxide CO2 (or liquid such as saline or sterile water H2O) high pressure system is connected to the apparatus (catheter) 10. The high pressure system can be a tank, a hospital unit, a cartridge, etc. or any other component that stores and injects the gas or liquid. A high pressure gauge 154 can optionally be provided to indicate the pressure within the storage device. The high pressure system also includes a pressure regulator 156 to reduce the pressure from the high pressure source. The regulator is adjustable to set the pressure to the desired amount, e.g., 0-3 psi (0-155 mm Hg). A pressure gauge 158 can be provided on the low pressure side to measure the pressure after reduced by the pressure regulator 156. After fluid is injected into the uterine cavity or the fluid is injected until the desired pressure is achieved inside of the uterine cavity with the pressure monitored by pressure gauge 162 positioned upstream of the manifold, the pressure valve 160 is turned off and the pressure is monitored by the pressure gauge 162 (see also gauge 174 of FIG. 16). If there is a pressure drop, this indicates that there is a leakage from the uterine cavity. On the other hand, if the pressure remains constant, this confirms there is no leakage from the uterine cavity and the cavity is sufficiently sealed for application of the therapeutic agent. Note the cavity integrity checks disclosed herein are preferably performed with the plug, e.g., cervical plug, plugging the canal or body space to close off the body space, i.e., create a closed volume. In addition or as an alternative, a flow meter can be provided to perform the cavity integrity check. That is, the meter can determine if flow ceases. If the flow in the uterine cavity of the gas or liquid ceases after a period of time because the cavity is full so it cannot accept more fluid, then it is confirmed there is no leakage. However, if flow continues, then it indicates there is a leakage of fluid from the uterus. Thus, as can be appreciated, instead of checking for pressure decay, fluid flow can be measured to check the integrity of the cavity. As a double check, both a flow meter and a pressure gauge could be utilized.

The manifold is in the form of a control such as a switch 170 which has three positions: 1) a neutral position (Position A) wherein the selector switch 170 is in the off position; 2) a second position (Position B) wherein the selector switch 170 is in a cavity integrity checking position; and 3) a third position (Position C) wherein the selector switch 170 is in a therapeutic agent (e.g. TCA) injection position. In Position A, the fluid line 164 from the pressure source is not in fluid communication with the tube connecting to the fluid channel within the shaft so there is no injection of pressurized fluid into the uterine cavity. In Position A, the fluid line is also not in fluid communication with the fluid line 166 for injection of the therapeutic agent so there is no injection of agent. In Position B, the fluid line 164 is fluidly connected to the tube connecting to the fluid channel so the pressurized fluid can be injected into the uterine cavity to perform the integrity check. In Position B, the fluid line is not in communication with the line 166 for injection of the therapeutic agent so there is no injection of the agent. In Position C, the fluid line 164 is in fluid communication with line 166 not line 164 for injection of pressurized fluid into the therapeutic agent storage device 172 to inject the agent under pressure into body cavity (preferably relatively low pressure but greater than if not pressurized) through the dispensing member and the perforations in the dispensing member into contact with the endometrium or through a distal opening in the catheter shaft in alternate embodiments. Note switching mechanisms (switches or other controls) can be provided with additional positions for purging, filling the cavity, dwell time, etc., for use with the systems of FIGS. 19-34 described below.’

The system as noted above also includes a pressure gauge 162 (or 174), positioned distal/downstream of the manifold to measure the pressure within the uterine cavity. This ensures the pressure within the cavity does not exceed a maximum level that could cause outflow from the cavity or damage to the cavity. The pressure gauge measures the pressure for injection of the therapeutic agent. That is, the pressure level is preset (e.g., at or below 50 mm Hg) for the cavity integrity check at a level where there is no leakage (to provide a Go or No-Go test), so it informs the user that the agent can be injected into the cavity at a pressure equal to or less than the measured fluid pressure (from the cavity check) without leakage or damage due to excess pressure. That is, the integrity cavity check also ensures the agent is applied at a safe pressure. Stated another way, the cavity checking feature applies the gas or liquid at a pressure where it is determined there is no leakage through perforations in the uterine wall or into the fallopian tubes or back via the cervical canal that is not fully plugged. With knowledge of this pressure, the therapeutic agent can be applied at the same pressure (or a lower pressure) to ensure no leakage of the agent.

It is also contemplated that the manual manifold (switch) described herein could be replaced by an automated system that switches the connection from one line to another or switches the opening and closing of the lines. Additionally, it is contemplated that the suction line can be designed to be controlled by the manifold or an automated system.

The system of FIG. 18 is identical to the system of FIG. 17 except for the placement of the manifold in the fluid line. That is, the switch 172 is upstream of the therapeutic agent so that the agent flows through the manifold. In all other respects, the features/components and functions of FIG. 17 are fully applicable to the system of FIG. 18 so for brevity are not repeated herein. The identical reference numerals of FIG. 17 are used for corresponding parts in FIG. 18.

In use of the systems of FIGS. 17 and 18 for performing for example chemical ablative endometrial ablation, the suction valve is first opened to apply a vacuum to the uterine cavity. Next, suction valve 150 is turned off and the expandable member is expanded in the uterine cavity by relative movement of the shaft and outer tube, utilizing for example the slidable actuator. The switch 170 is moved from the neutral position (Position A) to the second position (the cavity check position—Position B) and the valve 160 is moved to the open position to enable the pressurized fluid to flow into the uterine cavity. The pressure gauge 162 monitors the pressure in the manner described above. Then the pressure valve 160 is turned off, and the suction valve 150 is moved to the on position to evacuate the fluid from the uterine cavity. After evacuation, the suction valve 150 is returned to the off position and the switch 170 is moved to the third position (agent injection position—Position C). The valve 160 is turned back on so pressurized fluid can flow into the storage container containing the therapeutic agent and the agent is injected under pressure through the catheter and into the uterine cavity for chemical ablation. After the ablation procedure, the valve 160 is turned off to cease the flow of the chemical agent, and the suction valve 150 is turned back on to suction the remaining chemical agent from the uterine cavity. The suction valve is then turned off. The expandable member is returned to the collapsed position within the sheath by relative movement of the sheath and shaft and the apparatus is removed from the uterine cavity. Note the foregoing provides one example of the method of use. The system can be used to apply other agents and can be used in other body spaces.

FIGS. 19-34 illustrate alternate embodiments of the system of the present invention designed for injection and evacuation/aspiration of liquids and gas in and out of the body cavity. The system in some embodiments includes a catheter having an injection module and a delivery module that communicate with each other. In other alternate embodiments, both the injection module and delivery module could be integrated into a single device. If separate devices, the injection module and delivery module can be packaged together or alternatively packaged separately. An aspiration module in some embodiments can be integrated with the delivery module or with the injection module. In other alternate embodiments, a separate aspiration module is provided so that the system would include three separate modules—a delivery module, an injection module and an aspiration module, or alternatively two modules—an integrated delivery and injection module and a separate aspiration module.

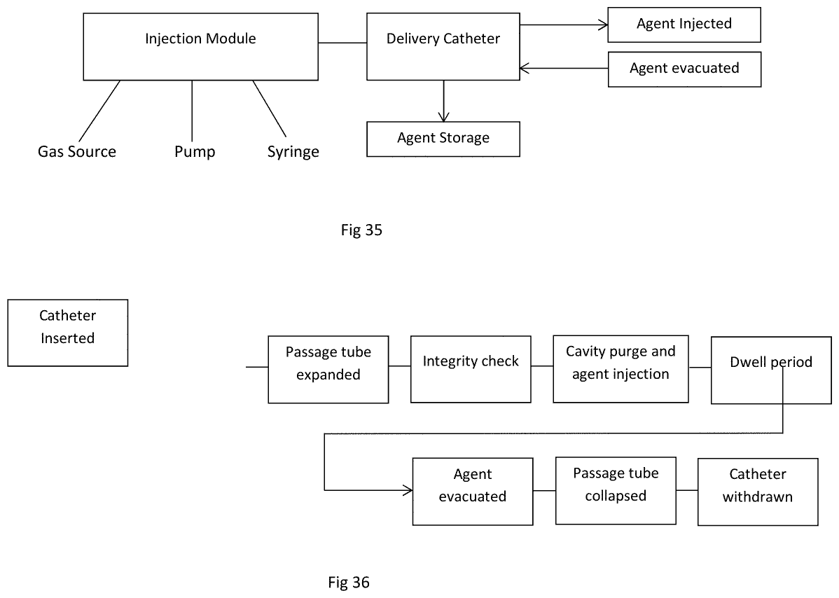

The injection module can be powered by a CO2 source, such a standard CO2 tank, CO2 line or a disposable CO2 cartridge to provide injection of the fluid at an increased pressure. Medical grade CO2 cartridges of different sizes can be utilized, for example 16 grams. Alternative sources of pressure, such as electrical, mechanical or manual pumps and syringes are also contemplated to inject pressurized fluid. Examples of these various power sources are shown in the block diagram of FIG. 35. Note that other fluids, i.e., gases/liquids, such as sterile water or saline, instead of CO2 can also be utilized as a power source to inject liquid at an increased pressure. As diagrammed in FIG. 35, the pressurized agent is delivered to the cavity via a delivery catheter such as the catheter of FIG. 19 discussed below and the agent is evacuated from the cavity through the catheter and into a storage unit.

In some clinical applications, it is advantageous that only the targeted tissue is exposed to the gas/liquid agent that is delivered by the system. For such applications, specifically for those that deliver the agent to a body cavity, a cavity integrity test is initially performed to confirm there is no leakage out of the cavity. Such integrity test is discussed above and is also utilized in the systems of FIGS. 19-37 and discussed below.

The systems of FIGS. 19-37 are discussed for delivery of TCA for chemical Global Endometrial Ablations (cGEA) treatment of Heavy Menstrual Bleeding (HMB). However, it should be understood that these systems (as well as the others systems and catheters discussed herein) could also be utilized in other clinical applications such as the clinical applications listed herein. The delivery module, e.g., the catheter and the expandable members, could have alternative configurations and sizes specifically suitable for the anatomy of various tissue targets.

The systems of the present invention provide a safe, effective, and easy-to-use cGEA therapeutic procedure by tightly controlling the TCA delivery and providing features that prevent accidental leakage, spillage or unintended exposure of healthy tissue. As noted above, the area of particular concern is leakage of TCA via a possible full-thickness uterine wall perforation or via the fallopian tubes. The system of the present invention prevents such leakage by testing the integrity of the uterine cavity before the TCA injection. By way of example, a typical pressure that would inject liquids into fallopian tubes is known to be 60+ mm Hg. If a user injects TCA at the pressure above the threshold that is patient specific, other areas could unintentionally be exposed to TCA.

One way to ensure the TCA pressure does not exceed the threshold pressure which would cause unwanted leakage in most of the patients is the provision in some embodiments of a pressure control system that includes components that regulate flow rates and/or pressure levels. The pressure control system can also include a source of pressure. In some embodiments, the injection module includes more than one pressure control system. For example, the module can include one pressure control system for the cavity integrity test and a separate pressure control system for the TCA injection. (The module can also include a separate control system for aspiration). In other embodiments, the system can use a single pressure control system for the cavity integrity check and the TCA injection. Once the body cavity is insufflated with CO2 at the predetermined pressure, the flow meter (or, alternatively, a flow sensor) provided in the system confirms that CO2 flow stops. This is a confirmation of the cavity integrity, i.e., there is no leakage of CO2 via a possible uterine wall perforation, fallopian tubes or into a vaginal cavity via a cervical canal. Another test to confirm cavity integrity is a pressure sensor to assess pressure within the cavity as discussed above. Once the cavity integrity is confirmed, TCA is injected at the same pressure. If CO2 doesn't leak, then TCA will not leak either at the same pressure. This assures that the injected TCA will only fill the uterine cavity, which is the target tissue of the proposed therapy. Note that the TCA can also be injected at a lower pressure than the C02 since if the CO2 doesn't leak at the higher pressure, the TCA won't leak at the lower pressure.

Another aspect of the cavity integrity check depends on the fluids used for the integrity check and for treatment (fluid meaning a liquid or gas). In preferred embodiments the fluid for the integrity check (referred to herein the “integrity check fluid”) has properties that make it easier to get into openings or perforations than the therapeutic or diagnostic agent. This prevents a situation where the integrity check detects no flow because the integrity check fluid cannot enter the openings but the fluid for the agent is able to pass through the openings and therefore leak through a uterine perforation or into the fallopian tubes or vaginal cavity. In some embodiments, to ensure this, the integrity check fluid utilized has a viscosity less than or equal to the viscosity of the agent and/or a surface tension less than or equal to the surface tension of the agent. Consequently, in these embodiments, by ensuring properties of the integrity check fluid and the agent are such that the agent does not more easily pass through perforations, an additional check is provided.

Preferably, the surface area of endometrial ablation should be greater than or equal to 90% of the total endometrial surface. This means it is desirable that TCA fills the cavity as much as possible and come in contact with at least 90% of its surface. A presence of air/gas bubbles/pockets inside of the cavity during the treatment might prevent proper tissue contact with and exposure to TCA. These bubbles or pockets could form from the air/gas that is present inside of the cavity itself and/or inside of the delivery catheter lines. To mitigate this, the system in preferred embodiments includes venting and/or aspiration capabilities to allow the air/gas bubbles to evacuate while TCA is being injected. Thus, aspiration and agent injection occur simultaneously in preferred embodiments. The air/gas bubbles can also be evacuated before TCA injection commences. In some embodiments, after purging bubbles during injection of the agent (TCA), aspiration is turned off and the agent is injected without aspiration for a pre-set period of time.

It is also contemplated that the pressure levels and flow rates for inflow/injection and outflow/aspiration could be controlled independently. Both injection and aspiration could therefore be used simultaneously or separately. The pressure levels and flow rates could be adjusted independently to achieve a needed inflow/outflow balance between injection and aspiration to lead to a desired result. For example, to purge the entire system of TCA after the TCA has been delivered to the uterine cavity, the pressure levels and flow rates on the outflow/suction (aspiration) side could be controlled or set to exceed the pressure levels and flow rates on the inflow/injection side. Conversely, if the pressure levels and flow rates on the inflow/injection side are controlled or set to be higher than on the outflow/suction side, the uterine cavity could be filled with TCA even if the suction is still operational. Other possible flow effects based on the pressure level and flow rate parameters set for injection and aspiration could include swirling and liquid/gas circulation through the cavity. Simultaneous operation of inflow/injection and outflow/venting/suction allows for air/gas bubbles evacuation and assures that the exposure of the endometrial surface to TCA is optimized/maximized.

To effect aspiration in the system, a source of vacuum, such a vacuum/suction pump, is provided. In addition to venting/aspirating/evacuating bubbles/air pockets, the aspiration system could also be used to facilitate outflow (evacuation) from the body cavity of gases and liquids, for example remaining/unused TCA, at the end of the procedure. It could also be used to evacuate the fluid used for the cavity integrity check. An electrical, mechanical or manual vacuum pump or syringe can be utilized. Alternatively, a Venturi pump could be powered by a pressure control system. In some embodiments, the Venturi pump could be powered by the same CO2 source that powers the integrity test and/or TCA injection. It is contemplated that the source(s) of pressure and vacuum could be located within the injection module, or alternatively, outside the injection module, e.g., mounted to or adjacent the catheter handle.

The system in accordance with some embodiments includes an injection module or console that controls pressure levels and flow rates of gases and liquids. The module includes a pressure regulator(s) and can further include a flow control adjustable or fixed orifice to restrict flow through the fluid line, i.e., restrict the flow of the low viscosity CO2 during the cavity integrity check. For a more precise pressure control, multiple pressure regulators could be arranged in series, such that they are gradually reducing pressure starting with a high pressure level from the CO2 source and down to a very low pressure at the last stage that is responsible for injection of gases/liquids into the uterine cavity. The pressure regulators control the integrity fluid pressure and therapeutic agent pressure. The module can also include sensors for measuring pressure at one or multiple times during injections. Regulators for aspiration could also be provided in or separate from the module.

A system that includes an injection module and a delivery catheter that are configured for cGEA for HMB will now be described by way of example. In this system, the sequence of procedural steps listed below is executed using a number of pinch valves that are located in the handle of the catheter to open and close the input and output fluid lines. However, other mechanical valve designs are also considered. It should be understood that alternatively, this sequence could be executed by flow and pressure controls that could be located inside of the injection module instead of outside the module, e.g., in or adjacent the device handle. Further, these control components could be activated manually or using an automated control system, such as electronic.

In some embodiments, the flow lines are shared to achieve multiple functions. For example, the inflow of CO2 for the integrity test and inflow of TCA for treatment can be effected through a common channel in the catheter shaft. Alternatively, individual delivery channels in the catheter shaft could be used—one channel for the integrity test and the other for the TCA. Also, as described herein, the inflow of CO2 and TCA can be via an internal diameter of a main shaft (through a single, or alternatively, two independent channels), while outflow/suction can occur via a perforated tube that is located at the catheter's distal end. It should be understood that this could be reversed so the inflow of CO2 and/or TCA are through the perforated tube and outflow/suction occurs via an internal diameter of the main shaft or in alternate embodiments, inflow and outflow are through the same passage and components. The system could be equipped with features for both venting and suction.

The inflow and outflow are preferably balanced so that during simultaneous injection of TCA and aspiration, the outflow is not too excessive so as to aspirate too much of the TCA before it can perform its ablation function but is sufficient to evacuate bubbles. That is, if the agent pressure is too high relative to the aspiration pressure, the air bubbles won't be able to exit and complete coverage of the endometrium by the TCA might not be achieved. Conversely, if the agent pressure is too low compared to the aspiration pressure, too much agent will be evacuated so complete coverage might not be achieved and the TCA might not be left in the cavity long enough.

The entire system or just the catheter or just the module could be disposable. Alternatively, the catheter and/or module can be reusable. When reusable modules are used, it is preferred that their components are not exposed to liquids or TCA. In the system that is described below, the injection module can be made as a reusable component since it controls gas flow of CO2 and suction, but no TCA flows through it.

Turning now to FIGS. 19-27, one embodiment of the catheter (delivery module) for delivering the agent, e.g., TCA, to the cavity is illustrated. The delivery catheter is designated generally by reference numeral 200 and includes a shaft assembly 212 extending distally of the handle assembly 230. The handle assembly 230 includes two handle halves, only the right handle half 230a is shown so the internal components are visible. The handle assembly 230 further includes a tube pinch valve assembly 234 and a cam plate 236 for opening and closing the fluid lines. A perforated tube 214 extends distally from the shaft 217. The shaft assembly 212 includes a tubular shaft 217 that is attached to a proximal flow fitting 218 that has three flow ports 218a, 218b and 218c. Port 218c is utilized for aspiration. The tube section 214a is attached at a proximal end to the port 218c such that inflow or outflow from the port 218c is exclusively interconnected and communicated with the internal channel of the tube 214. Port 218a is for agent delivery and tube 218b is for integrity checking fluid delivery. The flow ports 218a and 218b are optionally interconnected and communicate with the inside lumen of the shaft 217. The shaft 217 is covered by an axially slidable sheath 215. The sheath 215 is attached to a sheath hub 216 that is used for the sheath retraction to expose the perforated tube 214 at the distal end of the catheter 200. That is, the user grasps hub 216 and moves it proximally to move sheath 215 proximally to allow expansion of perforated tube 214 and moves hub 216 distally to move sheath 215 distally to cover and collapse the perforated tube 214. The sheath hub can include a proximal undercut 216a which interlocks with a distal section 218a of the fitting to secure the sheath in the retracted position and prevent accidental sliding forward of the sheath 215. The interlock can be a snap fit, frictional engagement, or other forms of securement. A cervical plug 219, which can be conically shaped as illustrated, although other configurations are also contemplated such as illustrated in the embodiment of FIG. 20A, is slidably mounted to the sheath 215 and slides over the sheath 215 to occlude the cervical canal of the uterus during the procedure. In FIG. 20A, an alternate embodiment of the cervical plug 219a is illustrated which has a series of elastomeric flexible discs 219b, 219c and 219d of different sizes as shown (progressively increasing in size in a proximal direction) which can be pushed further into the cervical canal as they flex to the shape of the canal. The flexibility enables the plug 219a to be pushed into the other side of the canal. In all other respects, the device is the same as FIG. 20A so is labeled with the same reference numerals. The plugs 219 and 219a can be used with the other catheters disclosed herein.

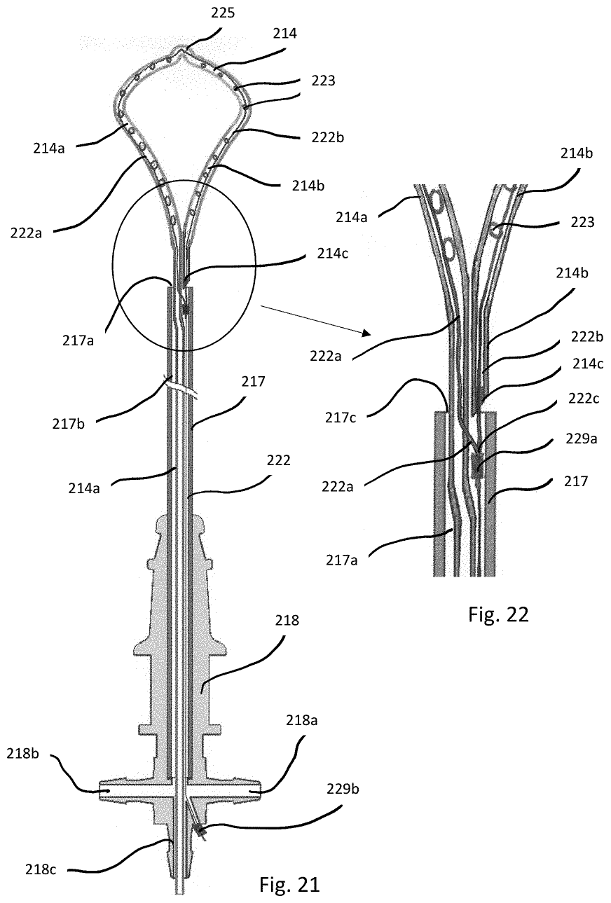

The perforated tube 214 is formed into a loop-shape at the distal end of the shaft assembly terminating at 235. As best shown in FIGS. 21 and 22, the tube 214 has two sections 214a and 214b. The tube section 214a extends from the proximal end to the distal end of the shaft assembly 212 and is located within the internal lumen 217b of the tubular shaft 217. The tube 214a extends past the distal edge 217a of shaft 217. The tube section 214b terminates the tube 214 just distally to the distal end 217a of the shaft 217 such that only the tube section 214a passes through the internal diameter (lumen 217b) of the shaft 17. This keeps the internal diameter of the shaft 217 more open to maximize flow of gas/liquid through its internal lumen 217b. This also minimizes the outer diameter of the shaft 217 since only one of the tube portions is positioned within the internal diameter. The end of the tube section 214b is occluded, for example by fusing it or blocking its internal channel with an adhesive 214c. Note the tube 214 in preferred embodiments is a single tube that loops around as shown in FIG. 21. However, in alternate embodiments, more than one tube can be used to form the loops as in FIG. 10A, for example. Different arrangements of the tube(s) are also contemplated.

The tube portions 214a, 214b which are exposed in the body cavity include a plurality of perforations (openings) 223 along the length. (Only a few of the openings 223 are labeled for clarity). The multiple perforations 223 allow inflow or outflow of liquids and gases to the uterine cavity. The number, size and spacing of the openings can vary from that shown so long as they are configured to achieve their functions as described herein.

Positioned inside of the tube 214 is a backbone wire 222. The wire 222 is preferably made from a shape memory material, such as Nitinol, although it can be made of alternative materials with sufficient spring like characteristics to expand the tube 214. It is pre-formed in a loop shape (its shape memorized state/condition) and acts as a spring forcing the compliant tube 214 to take the same shape. The wire 222 has two sections 222a and 222b. The end of the wire section 222a serves as a mechanical attachment of the end of the tube section 214b to the tube section 214a. The wire section 222a is mechanically attached, for example with a crimp ferrule 229a or welding, to the wire section 222b. The wire section 222b extends to the proximal section of the shaft assembly 212 where it is mechanically anchored to the fitting 218, for example, with a crimp ferrule 229b. In alternate embodiments, instead of the wire section 214b attached to the ferrule 229a at a distal region of the catheter, wire section 214b extends through the shaft lumen and is attached to wire section 214a at a proximal end. The spring action of the wire 222 effects opening of the loop of the tubular structure 214 when the sheath 215 is retracted to expose the loop from the confines of the wall of the sheath 215. The open loop facilitates delivery and distribution of liquid/gas throughout the uterine cavity and reduces dependence on pressure to achieve such distribution. When the loop opens, i.e., expands, its transverse dimension, defined as the distance across from tube section 214a to tube section 214b increases. The transverse dimension is represented by reference letter T in FIG. 21. Note the outer diameter of the tube does not necessarily expand in this embodiment (although in some embodiments it could), but the expansion is due to the change in the configuration of the loop i.e., the size of the loop increases.

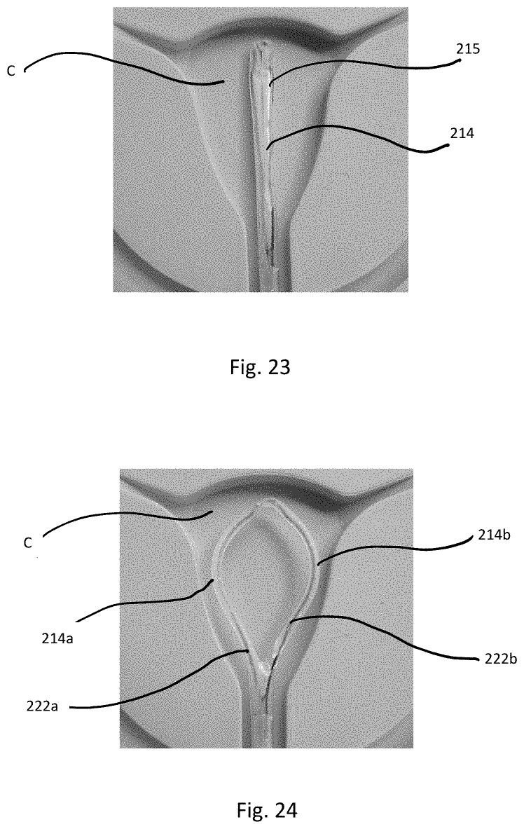

In use, for delivery, the tube portions 214a, 214b are in a reduced profile position within the sheath 215 which is in a distal position. Sheath 215 facilitates ease of insertion of the distal portion of the catheter 200 into a uterine cavity C. When the sheath 215 is advanced over the tube 214 (or the tube 214 withdrawn into the sheath 215), the backbone wire 222 is deformed/compressed from its loop-shaped free state and the distal section of the tube 214 is collapsed. After delivery, the sheath 215 is retracted to expose the tube portions 214a and 214b (or the tube 214 is advanced from the sheath 215), enabling the tube 214 to expand to its loop configuration of FIG. 21 due to the force of wire 222 for placement within the cavity C. At the end of the procedure, the sheath 215 is advanced to encapsulate and collapse the loop (or the tube 214 is withdrawn into the sheath 215) to enable withdrawal from the patient's body. Alternatively, the wire can collapse as the catheter is withdrawn from the patient's body. That is, in alternate embodiments, the wire has sufficient flexibility so that the catheter can be removed without first collapsing the wire in the cavity via the sheath as the wire would collapse as it is pulled out of the cavity. FIG. 23 shows the distal portion of the catheter 200 inserted into the uterine cavity C while the sheath 215 is in the advanced position. Once fully inserted, a user will pull on the hub 216 (shown in FIG. 19) and retract the sheath 215 as shown in FIG. 24. This allows the backbone wire 222 to return to its free state and open the loop at the end of the tube 214. The backbone wire 222 is configured to maximize the area of the loop relative to the area of the uterine cavity. Note it is also contemplated that the tube instead can be made of a material to expand to the looped configuration without an internal wire.

Note in the embodiment of FIG. 19, the TCA is injected through the lumen of the shaft 217 and exits the distal opening 217c into the body cavity and the evacuation is conducted through the perforations in the tube 214. The integrity check fluid is also conducted through the lumen of the shaft 217, exiting distal opening 217c. The same pressure source can be utilized. Alternate pressure sources can also be utilized.

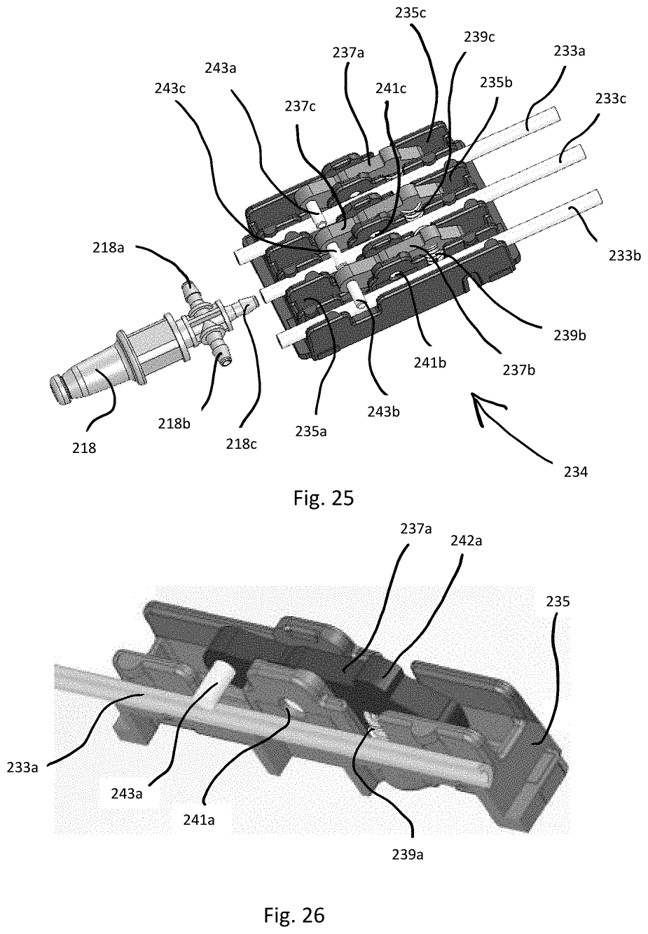

FIGS. 25-27 show one embodiment of the system that uses mechanical pinch valves to open and close the lines 233a, 233b and 233c that are connected to the flow ports 218a, 218b and 218c of the fitting 218. The assembly 234 is positioned at the handle housing 230 of catheter 200 and includes three pinch arms 237a, 237b and 237c (collectively pinch arms 237). Each pinch arm 237a, 237b, 237c pivots independently, so it selectively opens and closes each flow line when intended. Each pinch arm 237a, 237b, 237c pivots, respectively, around a pin 241a, 241b, 241c (collectively pins 241) relative to the respective pinch block 235a, 235b, 235c (collectively pinch blocks 235). The pin 241 extends through an opening in a side wall of the respective pinch block 235. Springs 239a, 239b, 239c bias the pinch arm 237a, 237b, 237c, respectively, into position where the pinch portions 243a, 243b, 243c of the respective pinch arm 237 compresses/pinches the tubes (also referred to as fluid lines) 233a, 233b, 233c (collectively tubes 233). This corresponds to the closed position of the fluid lines. As shown, each pinch portion 243 is in the form of a cylinder extending transversely (laterally) from the pinch arm 237 and is positioned over the tube 233 in the orientation of FIG. 26. The pinch portion 243 presses against the tube 233 to compress and close off the tube 233. In alternate embodiments, to conserve space, the pinch portions 243 can lie under the arms 237 or are oriented downwardly rather than transverse and/or the tubes 233 can lie under the pinch portions 243 rather than alongside laterally so the overall width of the pinch valve system is reduced.

In the embodiment of FIG. 25, fluid line 233c, connected to port 218c controls aspiration; fluid line 233a connected to port 218a controls TCA inflow and fluid line 233b connected to port 218b controls integrity check fluid inflow.

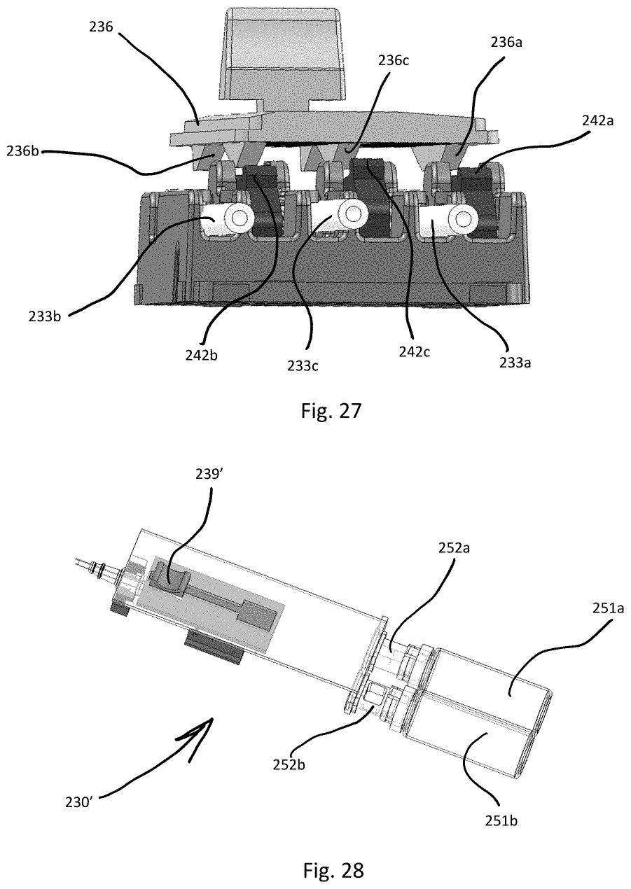

Pinch arms 237a, 237b, 237c have a guide surfaces 242a, 242b, 242c (collectively guide surface 242). Cam plate 236 (FIG. 27) has cam surfaces 236a, 236b and 236c. These cam surfaces 236a, 236b and 236c interact with corresponding guide surfaces 242a, 242b and 242c of the pinch arms 237a, 237b and 237c. The elevations of the cam surfaces 236a, 236b and 236c press on the guides surfaces 242a, 242b and 242c and create force that compresses springs 239. This pivots arms 237 away from tube 233 into position where they are no longer compress tubes 233 thereby opening the internal channels of the tubes for gas or liquid flow.

The cam surfaces 236a, 236b and 236c can be located so that they can open and close the lines 233a, 233b and 233c and execute the sequence of procedural steps described herein. For example, they can be staggered so they open and close the lines in the desired sequence when the actuator, e.g., lever, 239, is advanced. Alternatively, separate cam plates can be provided with separate actuators for each cam plate to selectively open and close the lines. As an alternative to a slidable actuator, a push button, a lever or other type of actuator(s) can be utilized to effect opening and closing of the valves to open and close the lines. Additionally, valves other than pinch valves can be utilized to open and close the lines as the pinch valves disclosed herein are one example of a mechanism to open the lines to allow flow and close the lines to prevent flow. The valves are preferably normally in the closed position requiring actuation to open the lines, but, alternatively, the valves can be normally in an open position so the fluid lines are open requiring actuation to close the valves to close the fluid lines.

Various forms of valves can be provided at the handle portion of the catheter mechanically controlled by the user to selectively open and close the fluid lines such as the pinch valves described above. In alternate embodiments, control of opening and closing the fluid lines can be at the injection module rather than at the catheter. Such controls can be manually (mechanically) applied at the injection module or alternatively electronically controlled. For example, the control module can have a regulator and a valve controlled by a solenoid to control the CO2 source. A switch to the TCA line from the CO2 line can be effected by a mechanically actuated valve or an electronically activated valve at the module. Note an electronically controlled valve or a manually controlled valve can also alternatively be utilized in an assembly outside the injection module, e.g., mounted to or adjacent the handle portion of the catheter.

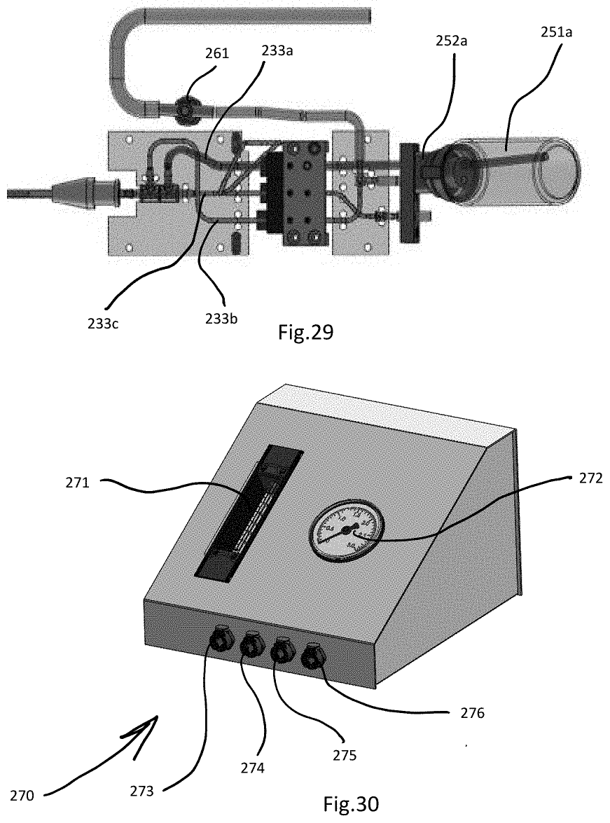

As shown in FIG. 29, a valve 261 can be provided on the fluid line that could be activated by pressing its button. This could offer an additional flow control to supplement the pinch valve mechanism. For example, the valve can independently open and close flow of CO2 from the injection module through integrity check fluid line 233b. Other supplemental valves on one or more fluid lines could also be provided.

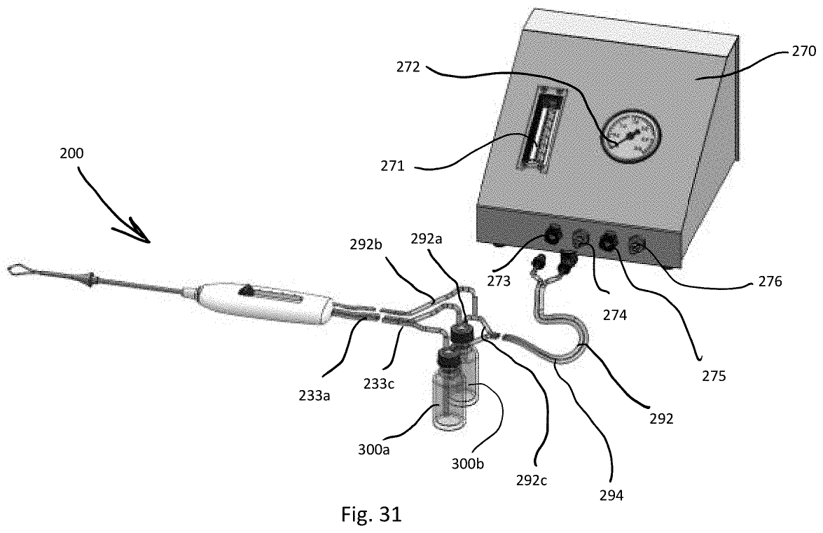

In embodiments having vials fluidly connected to the fluid lines, the vials (bottles) containing the fluid (for inflow and outflow) can be mounted to the catheter, e.g., mounted to the handle 230, or alternatively stand-alone vials not mounted to the handle (e.g., FIG. 31 discussed below). FIG. 28 illustrates an example wherein the vials are mounted to the handle housing of the catheter. Two vials 251a and 251b are attached and fluidly connected to the proximal portion of the handle assembly 230′ of the catheter (e.g., catheter 200) via connectors 252a and 252b, respectively. One of the vials, e.g., vial 251a, is used for storage of the liquid, e.g., TCA, that is intended for injection into the cavity for the therapeutic treatment. The second vial, e.g., vial 251b, is used for collection of liquid, e.g., TCA, after it already interacted with tissue in the procedure and is evacuated from the cavity. If necessary, this evacuated liquid could be collected, preserved and analyzed. In alternate embodiments, a third vial is provided which can be used to collect the liquid, e.g., sterile water or saline, if a liquid is used for the cavity integrity check instead of CO2. The catheter of FIG. 28 is identical to catheter 200 of FIG. 19 except for the handle housing which can support the vials 251a, 251b. Therefore, for convenience, the like components to FIG. 19 have in FIG. 28 been provided the same reference numerals except that the reference numerals of FIG. 28 have “prime” designations.

As an alternative to the vial or bottle for collecting the TCA from the body cavity, a bag such as a propylene bag can be used. The bag would be connected via a connecting tube to a connection port on the catheter handle, e.g., handle 230. Thus, in these embodiments, the bag is not mounted on the handle housing. Note the bag or vial containing the evacuated TCA would include a material therein to absorb and neutralize the acid for disposing and protection of the user.

One embodiment of an injection module separate from the delivery module (catheter) is shown in FIG. 30. Injection module 270 is equipped with a flow meter 271 to measure the amount of fluid passing through the fluid line and a pressure gauge 272 to measure fluid pressure. Injection module 270 can also have a vacuum gauge and second flow meter for aspiration. The readouts can be analog or digital. Module 270 also includes flow connectors 275 and 276. Additional flow connectors 275 and 276 could also be provided as shown in FIG. 31. In some embodiments, flow connectors 275 and 276 can be used for the Venturi pump discussed below. It is contemplated that a different number of flow connectors than shown can be provided to provide various functions. For example, the connector 273 could be used for connection of a CO2 source from the injection module 270 to the CO2 line 233b of the delivery catheter 200 for inflow of CO2 for the cavity check and for inflow of the agent under pressure. This connector 273 could also function as a check valve, such that when a CO2 line from the catheter 200 is not connected, the check valve is closed preventing unnecessary leakage of CO2. The connector 274, for example, could be used for suction and could be connected to the suction line 233c of the delivery catheter 200. If separate lines are used for the integrity check and agent, then, for example, connector 273 can be used for the integrity check and connector 275 for agent injection. Other possible uses of these connectors are contemplated.

FIG. 31 illustrates the catheter 200 being connected to the injection module 270. Bottles (vials) 300a, 300b are connected by tubes to ports 273, 274 of injection module 270. Tube 292 extends from connector 273 into TCA bottle 300b to inject CO2 into the bottle 300b and the pressurized agent from bottle 300b into fluid line 233a. Tube 292 is split at Y-connector 292c so tube 292a extends into the bottle 300b and tube 292b bypasses the bottle 300b and extends into the catheter to deliver CO2 to the cavity for the integrity check. Tube 294 extends from connector 274 and applies suction so the TCA after the procedure is suctioned into bottle 300a through fluid line 233c. At the back panel of the module 270 is an external suction port for vacuum and a port for connection of the CO2 source.

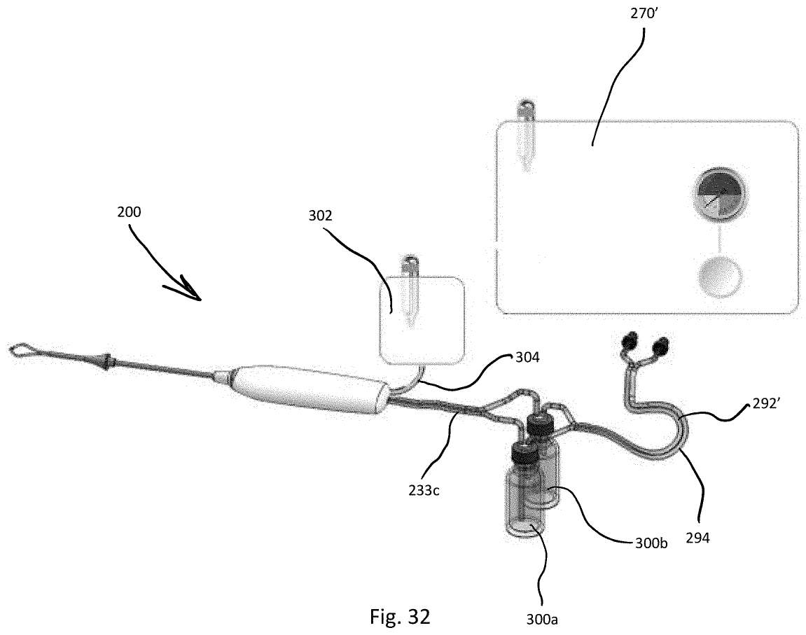

FIG. 32 is a view similar to FIG. 31 except showing a different CO2 source used for the cavity integrity check. CO2 source 302 is separate from the CO2 source used for injecting TCA through tube 292′. Tube 292′ is not split as in FIG. 30 but extends from a first CO2 source to the TCA bottle 300b. Tube 304 extends from another CO2 source 302 directly into the handle portion of the catheter 200. Tube 294 is the same as in FIG. 31. The module 270′ of FIG. 32 can have the flow meter(s) and gauge(s) as in the module of FIG. 31.

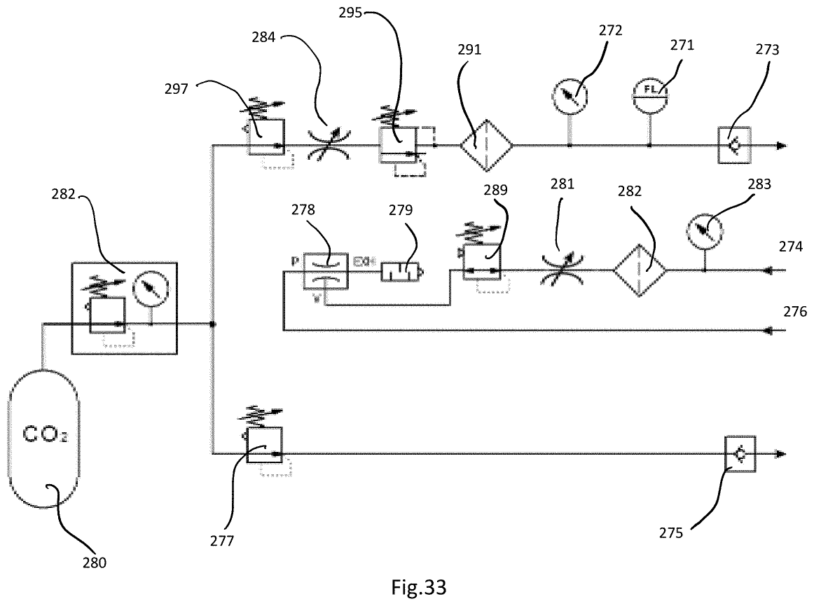

FIG. 33 shows a pneumatic diagram of the injection module 270 that is powered by a CO2 source and uses a Venturi vacuum pump. As explained above, other sources of pressure and vacuum and alternative means of their control are also contemplated.

The module 270 is equipped with a CO2 source 280 that is connected to a primary pressure regulator 282 and a primary pressure gauge. This regulator reduces the pressure from the CO2 source, e.g., from a pressure level that might exceed 3,000 (three thousand) psi down to below 50 psi level for example. A secondary pressure regulator 283 can be provided to further reduce pressure to a level appropriate for injection of the CO2 into the cavity. Preferably, this pressure level is below 1 psi or 50 mm Hg, although other pressure levels are also contemplated. An adjustable orifice 284 can be provided to provide additional control over the flow rate by restricting the flow of the low viscosity CO2 to increase the viscosity. (The TCA is more viscous so there is not the same need to restrict its flow out of the bottle). To provide a safety backup system, a pressure relief valve (PRV) 295 can be provided. The valve 295 can be set at a pressure level just over the pressure setting of the secondary regulator 297. For example, if the regulator 283 is set to 25 mm Hg, the pressure relief valve 295 can be set to 27-30 mm Hg. A gas filter 291 can be provided to prevent passage of particles into the catheter lines. The gas filter 291 can be located within the injection module 270 or alternatively outside of the module and can be disposable. A flow meter 271 is provided for the cavity integrity test described above. A pressure gauge 272 is an indicator of the pressure setting that is used for injection. The connector 273 could be used for connection of CO2 source from the injection module 270 to the CO2 line 233a of the delivery catheter 200.

In this embodiment, the Venturi vacuum pump is powered by the same CO2 source 280 and the primary pressure regulator 282. A secondary pressure regulator 277 can be provided in some embodiments to reduce pressure that powers the Venturi pump. The output of this regulator 277 is connected to the connector 274, which will in turn connect to a catheter line 233c which provides for aspiration. This connector 274 could also act as a check valve. The catheter line is controlled by a pinch valve that will open it when suction function is desired. When this line is open, the pressure from the regulator 277 is delivered to the Venturi pump 278 that can also be equipped with an exhaust 279 to reduce noise. Other components that could be provided include the PRV valve 289, adjustable flow orifice 281, a gas filter 282 and a vacuum gauge 283. The Venturi pump generates vacuum and is connected to the connector 276. With the provision of the Venturi pump which converts pressure into vacuum, a fluid line extends from connector 275 into the catheter handle and a second fluid line extends from the catheter handle into connector 276. To open and close off suction, rather than using for example a pinch valve to close tube 294 or fluid line 218c within the catheter handle, the CO2 supply to the Venturi pump is closed off to control the vacuum (aspiration).

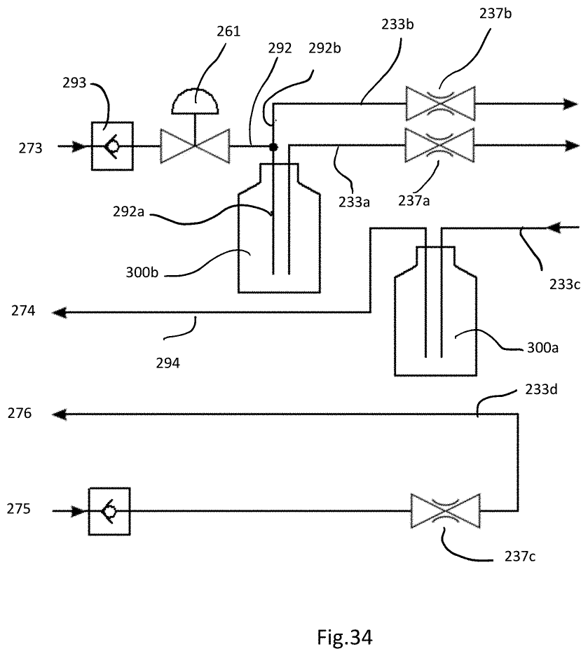

FIG. 34 is a pneumatic diagram of the catheter 200. The pressure line 292 is connected to the connector 273 of the injection module. The optional check valve 293 and the push-button valve 261 control pressure delivery from the injection module. When the check valve 274 and the push-button valve 261 are open, the CO2 lines 292 and 292a pressurize the vial 300b. The pinch valves 237b and 237a control the CO2 line 233b (and 292b) and the TCA line 233a, respectively.

The line 294 is connected to the connector 274 and could have an optional check valve. The line 294 is controlled by a pinch valve 237c that opens and closes a pressure supply to the Venturi pump via line 233d. When open, the vacuum generated by the pump creates a suction force within the vial 300b via a line 294 and pulls liquid out of the cavity through the line 233c.

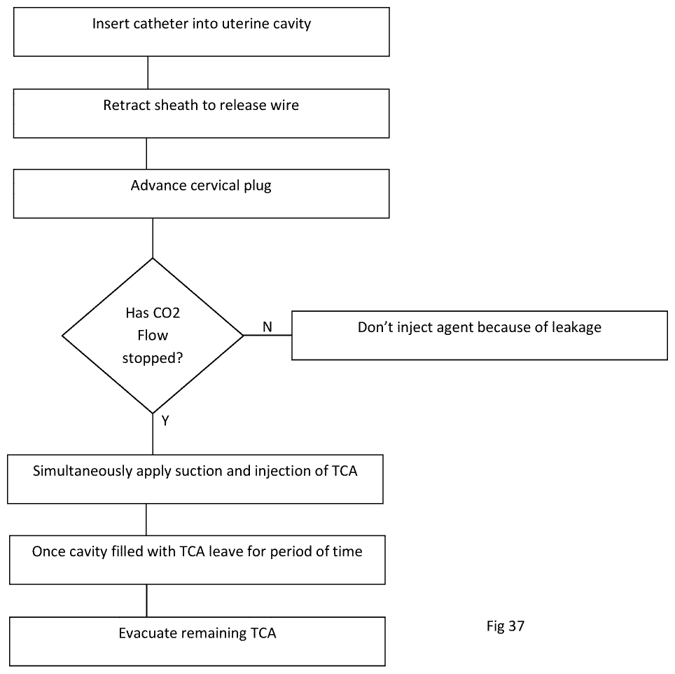

Some methods of the present invention using the injection module and delivery module include the following procedural steps. However, these steps are provided by way of example, as the procedure could include additional steps or omit some of the steps. The steps in one embodiment are as follows and are depicted in the diagrams of FIGS. 36 and 37.

The block diagram of FIGS. 36 and 37 show the overall sequence of steps. The catheter is inserted into the body cavity. The cervical plug is advanced. Once in position, the passage tube is expanded, e.g., by retraction of the sheath. Next pressurized fluid is injected for the cavity integrity check. (Note alternatively the cavity integrity check can occur prior to expansion of the passage tube). If the check integrity shows no leakage from (out of) the cavity, e.g., no leakage into the fallopian tubes, the cavity is purged and the therapeutic agent, e.g., TCA, is injected under pressure into the body cavity. The TCA is left in the cavity for a set period of time (the dwell period”). Then the TCA agent is evacuated from the cavity. The passage tube is collapsed and the catheter is withdrawn from the body cavity.

More specifically, the steps are as follows:
1. The catheter 100 is inserted into the uterine cavity C as shown on FIG. 23. The sheath 215 is retracted releasing the wire 222 from its compressed state and allowing the end of the perforated tube 214 to open into its loop configuration. The cervical plug 219 is advanced until it plugs the cervical canal.
2. An integrity check of the uterine cavity is performed (the “integrity check stage”). In this step, the cavity is insufflated with CO2 gas. Once the cavity is insufflated, the user utilizes the flow meter of the injection module to confirm zero-flow, which provides assurances that the CO2 gas doesn't leak via fallopian tubes, cervical canal or a possible perforation. If CO2 doesn't leak, then TCA will not leak either. If the flow meter indicates continued flow, then there is a leakage and TCA is not injected. At this point, the cervical plug can be adjusted and the cavity integrity check conducted again to confirm no leakage (zero flow) and assess whether TCA should be injected. If after adjustment, there is still flow, then TCA is not injected because of the presence of a leakage. If there is zero flow, TCA can be injected.
3. Suction and injection of TCA are simultaneously performed (with balanced flow as described herein) so that the system circulates TCA and, as a result, purges air and gas from the catheter lines and the cavity (the “bubble purging stage”). This step also pre-fills the cavity with TCA,
4. Once the cavity is filled, dwell time begins (the “dwell stage”). This allows tissue and TCA to continue interacting until the desired therapeutic effect is achieved.
5. The remaining TCA is evacuated via suction from the cavity and the lines.
6. The sheath 215 is advanced to compress the wire 222 and loop of tube 214, or the catheter is pulled back to collapse the wire, and the catheter 100 is withdrawn from the body cavity.

The system of the present invention in some embodiments can be conducted in a multi-step step timed process. In Step 1, a cavity integrity check is conducted by injection of pressurized fluid into the body cavity. The fluid is injected at a preselected pressure and the flow meter is checked to ensure flow stops. If flow stops, this indicates there is no leakage and the cavity is purged via application of aspiration/suction (Step 2) for a predetermined (preset) period of time. The time period for the purge can vary but for example can in some embodiments be between about 5 seconds to about 60 seconds and in more particular embodiments can be for example about 10 seconds to about 15 seconds (about or substantially as defined throughout this application means a deviation of (plus or minus) ±20%). The preset can include an indicator to prompt the user to terminate suction or alternatively there can be an automatic cutoff at the end of the pre-set time. In Step 3, pressurized TCA is applied to the cavity for a selected period of time. In some embodiments, the time is pre-set and can be for example between about 5 seconds to about 60 seconds and in more particular embodiments can be for example about 15 seconds to about 40 seconds. Other time periods for these various functions are also contemplated. In some embodiments it can be the same duration as the purge; in other embodiments it is a different duration. The time can be pre-set for a timed application of TCA, and the pre-set can include an indicator to prompt the user to terminate the TCA injection or alternatively there can be an automatic cutoff at the end of the pre-set time to terminate inflow of TCA. Step 4 is the “dwell step” or “dwell period” wherein the TCA is left in the cavity for a pre-set period of time for the ablation function. This preset can include an indicator to prompt the user to terminate the dwell period and initiate suction/evacuation or alternatively this suction/evacuation initiation can occur automatically at the end of preset time for the dwell step. In Step 5, the last step, the TCA is evacuated via suction from the cavity. This evacuation can be for a preset time period with either a prompt to terminate evacuation or an automatic cutoff of suction after a period of time.

In some embodiments, the foregoing steps in the procedure are user controlled, with the user determining when to move on to the next step. For example, the injection module can include a timer to provide an indication of the elapsed time for each step. The timer(s) can be on the handle, external of the catheter or on the injection module, and the output can be analog or digital. In such embodiments, the user watches the timer to determine when to end the step and initiate the next step in the procedure. In some embodiments, a visual indicator, such as a flashing light or an LED, and/or an audible indicator can indicate when the time has expired. In alternate embodiments, one or more of the steps are electronically controlled. That is, the timer(s) is electronically controlled such that at the expiration of the pre-set time, the system automatically terminates that step and initiates the next Step in the sequence without requiring human intervention. In such electronic systems, a digital flow meter can trigger an electric signal to launch the procedure sequence. If the flow equals zero, then the system automatically initiates/activates TCA injection. In some embodiments, launch of the TCA injection sequence is fully automatic; in other embodiments human intervention, e.g., pushing a button, initiates/activates the TCA injection.

Note a single source of pressure control can be utilized for the CO2 (integrity check) and the TCA (treatment agent). The CO2 goes into a pressure regulator and is split into a CO2 line and a TCA line. The advantage of a single line is it informs the user that there is no leakage at that pressure level. For example, if the calibration fails, the TCA will still be injected at the same level as the CO2.

In alternate embodiments two pressure regulators can be utilized. Associated with the CO2 cartridge would be one pressure regulator for the CO2 at a higher pressure, e.g., 25 mmHg and a second pressure regulator at a lower pressure for the TCA, e.g., at 20 mmHg.

In other alternate embodiments, two CO2 cartridges can be provided—one for the integrity check and one to inject pressurized TCA. More than two CO2 cartridges are also contemplated.

Although CO2 is described for the cavity check and pressurization of the agent, it is also contemplated that alternatively a pump, e.g., an air pump or a magnetic pump, can be utilized for the cavity check. Also, other fluids besides CO2 can be used for the cavity check such as sterile water.

The systems of the present invention could be used for various clinical applications where a controlled injection/insufflation of gas/liquid agents into a body space, for example a body cavity such as a uterine cavity, a lumen, a closed volume, etc. is required. One example of such application is a non-surgical lavage, which is washing out of a body cavity with water or other solutions, for example saline, antibiotics, chemotherapy agents, contrast agent, etc. In addition to the uterine cavity, this system could also be used for lavage of colon, stomach, or other body cavities. Another example is a wound irrigation for prevention of surgical site/wound infection and other gas/liquid agent delivery applications. Another application is an intravascular contrast agent injection.

Body cavity as used herein denotes a space in the body of a patient which includes a body lumen.

It is contemplated to add in some embodiments an element on the fluid input line that is not compatible with the chemical agent (e.g., TCA) so that it would degrade during the procedure to prevent reuse. For example, a cover or collar made of plastic can be positioned over a portion of the tube, for example, inside the handle, which plugs a side hole in the tube. As the plastic degrades, the side opening would become unplugged so that fluid would exit through the side opening. This would provide an indicator and prevent reuse of the system.

As noted above the apparatus described herein can provide a chemical global endometrium ablation device for the treatment of Heavy Menstrual Bleeding (HMB). The apparatus is a small profile non-invasive device that combines simplicity, cost effectiveness and ease-of-use of the IUD devices with the clinical efficacy of the global endometrial ablation (GEA) therapeutic approach making available for use in the office environment by OBGYN practitioners to treat HMB without learning any new skills. In some embodiments, the catheter can have an outer diameter of less than or equal to 4 mm, and is flexible and atraumatic and can eliminate the need for cervical dilations.

As noted above, the apparatus and systems disclosed herein are described by way of example for use for chemical ablation of the endometrium for endometrium ablation. However, the apparatus and systems disclosed herein can be used to apply other therapeutic agents to the uterine cavity as well as can also be used for injecting chemical ablative or other therapeutic agents to other regions or cavities of the body.

The apparatus and systems disclosed herein can also be used to inject diagnostic agents such as saline or sterile water for genomic language.

While the above description contains many specifics, those specifics should not be construed as limitations on the scope of the disclosure, but merely as exemplifications of preferred embodiments thereof. Those skilled in the art will envision many other possible variations that are within the scope and spirit of the disclosure as defined by the claims appended hereto.

What is claimed is:

1. An apparatus for delivering a therapeutic agent to a uterine cavity of a patient comprising:
a) an elongated member having a channel for passage of the agent into the uterine cavity of the patient for endometrial ablation, the channel having a distal opening; and
b) an expanding member extending distally of the elongated member the expanding member having a first condition for delivery and a second expanded condition for placement within the cavity, the expanding member having a plurality of perforations to provide a plurality of entrance openings for passage therein during aspiration of the cavity.

2. The apparatus of claim 1, wherein the expanding member comprises a tubular structure forming a loop distal of the elongated member in the second condition.

3. The apparatus of claim 1, wherein a fluid is injected into the uterine cavity prior to injection of the agent to assess the presence or absence of leakage in the uterine cavity, and the agent is injected at a pressure less than or equal to the pressure of injection of the fluid.

4. The apparatus of claim 1, wherein aspiration of the uterine cavity aspirates bubbles prior to injection of the agent and aspirates the agent after injection into the uterine cavity.

5. An apparatus for delivering an agent to a body cavity of a patient comprising:
a) a first passage for passage of the agent into the cavity of the patient, the first passage having an opening for exit of the agent; and
b) a second passage for aspirating the agent from the cavity;
c) wherein the agent is injected at an increased pressure and is injected simultaneously with aspiration of the cavity.

6. The apparatus of claim 5, wherein the second passage has a plurality of perforations to provide a plurality of entrance openings for passage of the agent into the apparatus during aspiration of the cavity.

7. The apparatus of claim 5, wherein the second passage aspirates bubbles from the cavity prior to injection of the agent.

8. The apparatus of claim 6, wherein a distal portion of the second passage has a looped configuration forming a loop, the loop having a first condition for delivery and a second expanded condition for placement within the uterine cavity.

9. The apparatus of claim 8, wherein the apparatus includes an elongated member having a lumen, and a tubular structure extends through the lumen and forms the looped configuration distal of the elongated member, wherein the tubular structure extends from the lumen and forms the loop terminating at an end which is distal of a distalmost edge of the elongated member.

10. The apparatus of claim 5, wherein a fluid is injected into the cavity prior to injection of the agent to assess the presence or absence of leakage out of the cavity, and the agent is injected at a pressure less than or equal to the pressure of injection of the fluid.

11. The apparatus of claim 10, wherein the fluid is injected through the first passage.

12. The apparatus of claim 10, wherein the fluid has one or both of a surface tension less than a surface tension of the agent and a viscosity less than a viscosity of the agent.

13. The apparatus of claim 5, further comprising a line connectable to a module, the module controlling a time period of injection of the agent so the agent in injected for a preset period of time.

14. The apparatus of claim 5, in combination with an injection module, the injection module includes a pressure controller to control pressure and a timer to control a time period of injection of the agent.

15. The apparatus of claim 14, wherein the injection module automatically transitions to injection of the agent if the absence of a leakage is assessed.

16. A method for injecting a therapeutic agent into a cavity of a patient comprising:
a) checking the integrity of the cavity to determine if there is leakage from the cavity;
b) aspirating the cavity to remove gas bubbles; and
c) injecting the therapeutic agent into the uterine cavity under controlled pressure simultaneously with aspirating the cavity.

17. The method of claim 16, wherein the agent is injected into the cavity for a pre-set period of time.

18. The method of claim 16, further comprising the steps of a) leaving the agent in the cavity for a preset period of time after injection into the cavity; and b) after the pre-set period of time evacuating the agent from the body cavity.

19. The method of claim 16, wherein if no leakage is determined by checking the integrity of the cavity, injection of the agent is automatically initiated.

20. The method of claim 16, wherein if no leakage is determined by checking the integrity of the cavity, a user actuates a valve to open a fluid line for injection of the agent.
